(19) United States
(12) Patent Application Publication
(10) Pub. No.: US 2018/0065933 A1
(43) Pub. Date: Mar. 8, 2018

(54) DEUTERIUM-ENRICHED HYPOXIA-INDUCIBLE FACTOR PROLYL HYDROXYLASE ENZYME INHIBITORS

(71) Applicant: Roger HANSELMANN, Lexington, MA (US)

(72) Inventor: Roger HANSELMANN, Lexington, MA (US)

(73) Assignee: Akebia Therapeutics, Inc., Cambridge, MA (US)

(21) Appl. No.: 15/559,200

(22) PCT Filed: Mar. 18, 2016
(86) PCT No.: PCT/US16/23132

§ 371 Date: Sep. 18, 2017

Related U.S. Application Data
(60) Provisional application No. 62/136,078, filed on Mar. 20, 2015.

(51) Int. Cl. C07D 213/81; C07B 59/00

(52) U.S. Cl. CPC C07D 213/81; C07B 2200/05; C07B 59/002

(57) ABSTRACT

Provided herein are deuterium-enriched compounds of Formula (I), Formula (II), Formula (III), Formula (IV), Formula (V), and Formula (VI). Pharmaceutical compositions comprising the isotope-enriched compounds, and methods of using such compounds are also provided.

This application claims the benefit of U.S. Provisional Application No. 62/136,078, filed Mar. 20, 2015, the entire contents of which are incorporated herein by reference.

### 1. FIELD

This disclosure relates to deuterium-enriched isotopologues of hypoxia-inducible factor (“HIF”) prolyl hydroxylase enzyme inhibitors, pharmaceutical compositions containing the same, and methods of using the same.

### 2. BACKGROUND

Hypoxia-inducible factor (HIF) is a transcription factor that is a key regulator of responses to hypoxia. In response to hypoxic conditions, i.e., reduced oxygen levels in the cellular environment, HIF upregulates transcription of several target genes, including those encoding erythropoietin. HIF is a heteroduplex comprising an alpha and beta subunit. While the beta subunit is normally present in excess and is not dependent on oxygen tension, the HIF-alpha subunit is only detectable in cells under hypoxic conditions. In this regard, the accumulation of HIF-alpha is regulated primarily by hydroxylation at two proline residues by a family of prolyl hydroxylases known as HIF prolyl hydroxylases, wherein hydroxylation of one or both of the proline residues leads to the rapid degradation of HIF-alpha. Accordingly, inhibition of HIF prolyl hydroxylase results in stabilization and accumulation of HIF-alpha (i.e., the degradation of HIF-alpha is reduced), thereby leading to an increase in the amount of HIF-alpha available for formation of the HIF heterodimer and upregulation of target genes, such as the Erythropoietin gene. Conversely, activation of HIF prolyl hydroxylase results in destabilization of HIF-alpha (i.e., the degradation of HIF-alpha is increased), thereby leading to a decrease in the amount of HIF-alpha available for formation of the HIF heterodimer and downregulation of target genes, such as VEGF.

The family of hypoxia inducible factors includes HIF-1-alpha, HIF-2-alpha, and HIF-3-alpha.

A new class of prolyl hydroxylase inhibitors and their use to treat or prevent diseases ameliorated by modulation of hypoxia-inducible factor (HIF) prolyl hydroxylase are described in U.S. Pat. No. 7,811,595, which is incorporated herein by reference in its entirety. The synthesis of such prolyl hydroxylase inhibitors is described in U.S. Patent Publication No. 2012/0309977, which is incorporated herein by reference in its entirety. Such compounds inhibit HIF prolyl hydroxylase, thereby stabilizing HIF-alpha. As a consequence of stabilizing HIF-alpha, endogenous erythropoietin (EPO) production is increased. As with all drugs, proper doses and dosing regimens for treating patients having diseases such as anemia are essential for achieving a desired or optimal therapeutic effect without adverse effects or unwanted side-effects. Indeed, many active compounds fail in clinical trials because an effective and safe dosing regimen cannot be found.

Therefore, a need exists for safe, effective, and non-toxic doses and dosing regimens that either avoid or reduce adverse or unwanted effects, provide an optimal therapeutic effect or both, that is, provide a desirable therapeutic profile.

Isotopic enrichment (e.g., deuteration) of pharmaceuticals to improve pharmacokinetics (“PK”), pharmacodynamics (“PD”), and toxicity profiles, has been demonstrated previously with some classes of drugs. (See, e.g., Lijinsky et. al., Food Cosmet. Toxicol.,Vol. 20, No. 4, p. 393 (1982); Lijinsky et. al., J. Nat. Cancer Inst., Vol. 69, No. 5, p. 1127 (1982); Mangold et. al., Mutation Res. Vol. 308, No. 1, p. 33 (1994); Gordon et. al., Drug Metab. Dispos., Vol. 15, p. 589 (1987); Wade D, Chem. Biol. Interact. Vol. 117, p. 191 (1999)).

Deuterium is a stable and non-radioactive isotope of hydrogen with an atomic mass that is double that of hydrogen (2.01355 amu and 1.0078 amu, respectively). It contains one proton and one neutron in its nucleus and has a natural abundance of 0.015%. Replacement of an atom for deuterium may often result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g, Foster et al., Adv. Drug Res., Vol. 14, pp. 1-36 (1985); Kushner et al., Can. J. Physiol. Pharmacol., Vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as fifty or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

### 3 SUMMARY

This disclosure relates to deuterium-enriched isotopologues of hypoxia-inducible factor (“HIF”) prolyl hydroxylase enzyme inhibitors, pharmaceutical compositions containing the same, and methods of using the same. In one embodiment, the isotopologue is a deuterium-enriched compound of Formula (II):

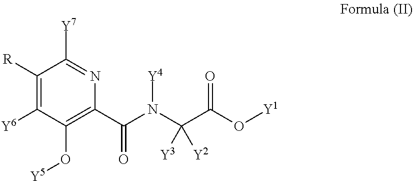

or a pharmaceutically acceptable salt, solvate or hydrate thereof, wherein

R is selected from:
(i) Y⁸; or
(ii) substituted or unsubstituted phenyl;

said substitution selected from:
(i) C(Y⁹⁻¹¹)₃;
(ii) halogen;
(iii) cyano;
(iv) Y¹²;
(v) Y¹³;
(vi) Y¹⁴;
(vii) Y¹⁵; or
(vii) Y¹⁶;

wherein one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and/or Y¹⁶ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and/or Y¹⁶ are non-enriched hydrogen atoms.

In certain embodiments, the isotopologue is a deuterium-enriched compound of Formula (III):

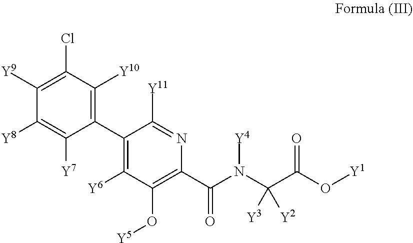

or a pharmaceutically acceptable salt, solvate, hydrate, or stereoisomer thereof, wherein one or more of Y¹, Y², Y³, Y⁴, Y⁵, Y⁶, Y⁷, Y⁸, Y⁹, Y¹⁰, and/or Y¹¹ is a hydrogen that is isotopically enriched with deuterium, and the others of Y¹, Y², Y³, Y⁴, Y⁵, Y⁶, Y⁷, Y⁸, Y⁹, Y¹⁰, and/or Y¹¹ are non-enriched hydrogen atoms.

In certain embodiments, the isotopologue is a deuterium-enriched compound of Formula (IV):

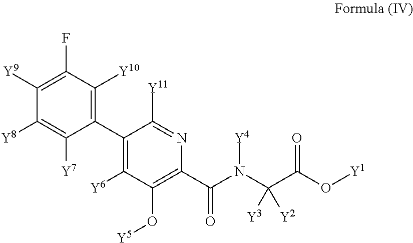

or a pharmaceutically acceptable salt, solvate, hydrate, or stereoisomer thereof, wherein one or more of Y¹, Y², Y³, Y⁴, Y⁵, Y⁶, Y⁷, Y⁸, Y⁹, Y¹⁰, and/or Y¹¹ is a hydrogen that is isotopically enriched with deuterium, and the others of Y¹, Y², Y³, Y⁴, Y⁵, Y⁶, Y⁷, Y⁸, Y⁹, Y¹⁰, and/or Y¹¹ are non-enriched hydrogen atoms.

In certain embodiments, the isotopologue is a deuterium-enriched compound of Formula (V):

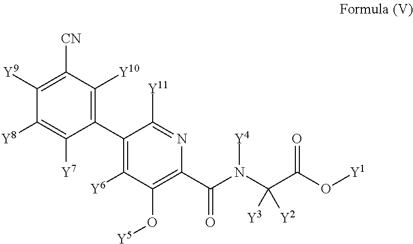

or a pharmaceutically acceptable salt, solvate, hydrate, or stereoisomer thereof, wherein one or more of Y¹, Y², Y³, Y⁴, Y⁵, Y⁶, Y⁷, Y⁸, Y⁹, Y¹⁰, and/or Y¹¹ is a hydrogen that is isotopically enriched with deuterium, and the others of Y¹, Y², Y³, Y⁴, Y⁵, Y⁶, Y⁷, Y⁸, Y⁹, Y¹⁰, and/or Y¹¹ are non-enriched hydrogen atoms.

In certain embodiments, the isotopologue is a deuterium-enriched compound of Formula (VI):

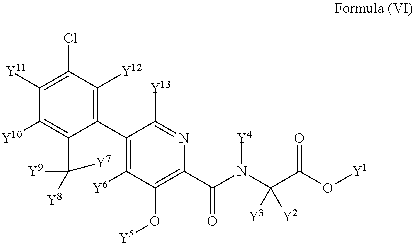

or a pharmaceutically acceptable salt, solvate, hydrate, or stereoisomer thereof, wherein one or more of Y¹, Y², Y³, Y⁴, Y⁵, Y⁶, Y⁷, Y⁸, Y⁹, Y¹⁰, Y¹¹, Y¹², and/or Y¹³ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², and/or Y¹³ are non-enriched hydrogen atoms.

### 4 DETAILED DESCRIPTION

The descriptions of the terminology provided below apply to the terms as used herein and unless otherwise specified.

### 4.1 Definitions and Abbreviations

The term “isotopically enriched” refers to an atom of a specific position of a compound having an isotopic composition other than the natural isotopic composition of that atom. “Isotopically enriched” can also refer to a compound containing at least one atom having an isotopic composition other than the natural isotopic composition of that atom. As used herein, an “isotopologue” is an isotopically enriched compound.

The term “isotopic enrichment” refers to the percentage of incorporation of an amount of a specific isotope at a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

As used herein, an “alkyl” group is a saturated straight chain or branched non-cyclic hydrocarbon having, for example, from 1 to 12 carbon atoms, 1 to 9 carbon atoms, 1 to 6 carbon atoms, 1 to 4 carbon atoms, or 2 to 6 carbon atoms. Representative alkyl groups include -methyl, -ethyl, -n-propyl, -n-butyl, -n-pentyl and -n-hexyl; while branched alkyls include -isopropyl, -sec-butyl, iso-butyl, tert-butyl, iso-pentyl, 2-methylpentyl, 3-methylpentyl, 4-methylpentyl, 2,3-dimethylbutyl and the like.

C₁₋₆ alkyl units include the following non-limiting examples: methyl (C₁), ethyl (C₂), n-propyl (C₃), iso-propyl (C₃), n-butyl (C₄), sec-butyl (C₄), iso-butyl (C₄), tert-butyl (C₄), n pentyl (C₅), tent pentyl (C₅), neo pentyl (C₅), iso pentyl (C₅), sec pentyl (C₅), 3 pentyl (C₅), n-hexyl (C₆), iso-hexyl (C₆), neo-hexyl (C₆), 3-methylpentyl (C₆), 4-methylpentyl (C₆), 3-methylpentan-2-yl (C₆), 4-methylpentan-2-yl (C₆), 2,3-dimethylbutyl (C₆), 3,3-dimethylbutan-2-yl (C₆), 2,3-dimethylbutan-2-yl (C₆), and the like.

As used herein, an “alkenyl” group is a partially unsaturated straight chain or branched non-cyclic hydrocarbon containing at least one carbon-carbon double bond and having, for example, from 1 to 6 carbon atoms. Representative alkenyl groups include propenyl and the like.

As used herein, an “alkynyl” group is a partially unsaturated straight chain or branched non-cyclic hydrocarbon containing at least one carbon-carbon triple bond and having, for example, from 2 to 6 carbon atoms. Representative alkynyl groups include propynyl, butynyl and the like.

As used herein, an “alkoxy” group is an alkyl-O— group in which the alkyl group is as defined herein. Representative alkoxy groups include methoxy, ethoxy, n-propoxy, isopropoxy and n-butoxy.

As used herein, an “cycloalkyl” group is a saturated cyclic alkyl group of from 3 to 6 carbon atoms having a single cyclic ring. Representative cycloalkyl groups include cyclopropyl, cyclobutyl, and cyclopentyl.

As used herein, an “cycloalkenyl” group is a partially unsaturated cyclic alkyl group containing at least one carbon-carbon double bond and from 3 to 6 carbon atoms having a single cyclic ring. Representative cycloalkenyl groups include cyclopropenyl and cyclobutenyl.

As used herein, a “cycloalkoxy” group is a cycloalkyl-O— group in which the cycloalkyl group is as defined herein. Representative cycloalkoxy groups include cyclopropyloxy, cyclobutyloxy and cyclopentyloxy.

As used herein, a “deuterium” group is a stable isotope of hydrogen having one proton and one neutron.

With regard to the compounds provided herein, when a particular atomic position is designated as having deuterium or “D,” it is understood that the abundance of deuterium at that position is substantially greater than the natural abundance of deuterium, which is about 0.015%.

As used herein, a “haloalkyl” group is an alkyl group as defined herein above with one or more (e.g., 1 to 5) hydrogen atoms are replaced by halogen atoms. Representative haloalkyl groups include CF₃, CHF₂, CH₂F, CCl₃, CF₃CH₂CH₂ and CF₃CF₂.

As used herein, a “halocycloalkyl” group is a cycloalkyl group as defined herein above with one or more (e.g., 1 to 5) hydrogen atoms are replaced by halogen atoms. Representative halocycloalkyl groups include 2,2-difluorocyclopropyl, 2,2-dichlorocyclopropyl, 2,2-dibromocyclopropyl, tetrafluorocyclopropyl, 3,3-difluorocyclobutyl and 2,2,3,3-tetrafluorocyclobutyl.

As used herein, a “heterocycloalkyl” group is a saturated ring of 4 to 7 atoms, preferably 5 or 6 ring atoms, wherein 1 or 2 ring members are selected from the group consisting of O, S and NR and the remaining atoms are carbon. There are no adjacent oxygen and/or sulfur atoms in the rings. Representative heterocycloalkyl groups are piperidyl, pyrrolidinyl, piperazinyl, morpholinyl, thiomorpholinyl, thiazolidinyl, 1,3-dioxolanyl, 1,4-dioxanyl, oxazolinyl, tetrahydrofuranyl, tetrahydrothiophenyl and tetrahydrothiopyranyl.

As used herein, an “aryl” group is an aromatic monocyclic or multi-cyclic ring system comprising 4 to 10 carbon atoms. Representative aryl groups include phenyl and naphthyl.

As used herein, a “heteroaryl” is a single ring, bicyclic or benzofused heteroaromatic group of 5 to 10 atoms comprised of 2 to 9 carbon atoms and 1 to 4 heteroatoms independently selected from the group consisting of N, O and S, provided that the rings do not include adjacent oxygen and/or sulfur atoms. N-oxides of the ring nitrogens are also included. Representative single-ring heteroaryl groups include pyridyl, oxazolyl, isoxazolyl, oxadiazolyl, furanyl, pyrrolyl, thienyl, imidazolyl, pyrazolyl, tetrazolyl, thiazolyl, isothiazolyl, thiadiazolyl, pyrazinyl, pyrimidyl, pyridazinyl and triazolyl. Representative bicyclic heteroaryl groups are naphthyridyl (e.g., 1, 5 or 1, 7), imidazopyridyl, pyridopyrimidinyl and 7-azaindolyl. Representative benzofused heteroaryl groups include indolyl, quinolyl, isoquinolyl, phthalazinyl, benzothienyl (i.e., thianaphthenyl), benzimidazolyl, benzofuranyl, benzoxazolyl, benzisoxazolyl, benzothiazolyl and benzofurazanyl. All positional isomers are contemplated, e.g., 2-pyridyl, 3-pyridyl and 4-pyridyl.

The compounds disclosed herein include all enantiomeric forms, diastereomeric forms, salts, tautomers, and the like.

The compounds disclosed herein include all salt forms, for example, salts of both basic groups, inter alia, amines, as well as salts of acidic groups, inter alia, carboxylic acids. The following are non-limiting examples of anions that can form pharmaceutically acceptable salts with basic groups: chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, citrate, and the like. The following are non-limiting examples of cations that can form pharmaceutically acceptable salts of the anionic form of acidic substituent groups on the compounds described herein: sodium, lithium, potassium, calcium, magnesium, zinc, bismuth, and the like. The following are non-limiting examples of cations that can form pharmaceutically acceptable salts of the anionic form of phenolic, aryl alcohol, or heteroaryl alcohol substituent groups on the compounds described herein: sodium, lithium, and potassium.

As used herein, the term “pharmaceutically acceptable salt” refers to a salt prepared from pharmaceutically acceptable non-toxic acids or bases including inorganic acids and bases and organic acids and bases. Suitable pharmaceutically acceptable base addition salts for a compound having a structure of Formula (I), Formula (II), Formula (III), Formula (IV), Formula (V), or of Formula (VI) or a compound selected from Metabolite 1 to Metabolite 2 but are not limited to, sodium, lithium, potassium, calcium, magnesium, zinc, bismuth, ammonium (including alkyl substituted ammonium), amino acids (e.g., lysine, ornithine, arginine, or glutamine), tromethamine, and meglumine. Suitable non-toxic acids include, but are not limited to, inorganic and organic acids such as acetic, alginic, anthranilic, benzenesulfonic, benzoic, camphorsulfonic, citric, ethanesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Other examples of salts are well known in the art, see, e.g., Remington's Pharmaceutical Sciences, 22nd ed., Pharmaceutical Press (2012).

As used herein, the term “hydrate” means a compound provided herein or a pharmaceutically acceptable salt thereof, that further includes a stoichiometric or non-stoichiometric amount of water bound by non-covalent intermolecular forces.

As used herein, the term “solvate” means a compound provided herein or a pharmaceutically acceptable salt thereof, that further includes a stoichiometric or non-stoichiometric amount of a solvent, other than water, bound by non-covalent intermolecular forces.

The phrase “an enantiomer or a mixture of enantiomers thereof; or a pharmaceutically acceptable salt, solvate, hydrate, or stereoisomer thereof” has the same meaning as the phrase “an enantiomer or a mixture of enantiomers of the compound referenced therein; a pharmaceutically acceptable salt, solvate, hydrate, or stereoisomer of the compound referenced therein; or a pharmaceutically acceptable salt, solvate, hydrate, or stereoisomer of an enantiomer or a mixture of enantiomers of the compound referenced therein.”

As used herein, the terms “prevent,” “preventing” and “prevention” are art-recognized, and when used in relation to a condition, such as a local recurrence (e.g., pain), a disease such as cancer, a syndrome complex such as heart failure or any other medical condition, is well understood in the art, and includes administration of a compound provided herein or a pharmaceutically acceptable salt, solvate or hydrate thereof, which reduces the frequency of, or delays the onset of, symptoms of a medical condition in a subject relative to a subject which does not receive the composition.

As used herein, the terms “treat,” “treating,” and “treatment” refer to the reversing, reducing, or arresting the symptoms, clinical signs, and underlying pathology of a condition in manner to improve or stabilize a subject's condition. The terms “treat” and “treatment” also refer to the eradication or amelioration of the disease or symptoms associated with the disease. In certain embodiments, such terms refer to minimizing the spread or worsening of the disease resulting from the administration of a compound provided herein or a pharmaceutically acceptable salt, solvate or hydrate thereof to a patient with such a disease.

In certain embodiments, the term subject or patient can refer to a mammal, such as a human, mouse, dog, donkey, horse, rat, guinea pig, bird, or monkey. In specific embodiments, a subject or a patient is a human subject or patient.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

With regard to the compounds provided herein, when a particular atomic position is designated as having deuterium or “D,” it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom.

It is understood that one or more deuteriums may exchange with hydrogen under physiological conditions.

The isotopic enrichment and isotopic enrichment factor of the compounds provided herein can be determined using conventional analytical methods known to one of ordinary skill in the art, including mass spectrometry and nuclear magnetic resonance spectroscopy.

### 4.2 Compounds

In certain embodiments, a deuterium-enriched HIF prolyl hydroxylase inhibitor or HIF-alpha stabilizer has a structure of Formula (I):

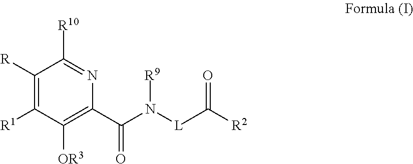

or a pharmaceutically acceptable salt, solvate or hydrate thereof, wherein

R and R¹ are each independently selected from:
(i) hydrogen
(ii) substituted or unsubstituted phenyl; or
(iii) substituted or unsubstituted heteroaryl;

said substitution selected from:
(i) C₁-C₄ alkyl;
(ii) C₃-C₄ cycloalkyl;
(iii) C₁-C₄ alkoxy;
(iv) C₃-C₄ cycloalkoxy;
(v) C₁-C₄ haloalkyl;
(vi) C₃-C₄ halocycloalkyl;
(vii) halogen;
(viii) cyano;
(ix) NHC(O)R⁴;
(x) C(O)NR^{5a}R^{5b}; and
(xi) heteroaryl; or
(xii) two substituents are taken together to form a fused ring having from 5 to 7 atoms;

R⁴ is a C₁-C₄ alkyl or C₃-C₄ cycloalkyl;

R^{5a} and R^{5b} are each independently selected from:
(i) hydrogen;
(ii) C₁-C₄ alkyl;
(iii) C₃-C₄ cycloalkyl; or
(iv) R^{5a} and R^{5b} are taken together to form a ring having from 3 to 7 atoms;

R² is selected from:
(i) OR⁶;
(ii) NR^{7a}R^{7b}; and

R⁶ is selected from hydrogen and C₁-C₄ alkyl or C₃-C₄ cycloalkyl;

R^{7a} and R^{7b} are each independently selected from:
(i) hydrogen;
(ii) C₁-C₄ alkyl or C₃-C₄ cycloalkyl; or
(iii) R^{7a} and R^{7b} are taken together to form a ring having from 3 to 7 atoms;

R³ is selected from hydrogen, methyl, and ethyl;

L is a linking unit having a structure —[C(R^{8a}R^{8b})]ₙ—

R^{8a} and R^{8b} are each independently selected from hydrogen, methyl and ethyl;

n is an integer from 1 to 3; and

R⁹ is selected from hydrogen and methyl,

wherein at least one hydrogen is replaced by a hydrogen isotopically enriched with deuterium.

In certain more specific embodiments, the deuterium-enriched HIF prolyl hydroxylase inhibitor or HIF-alpha stabilizer has a structure of Formula (II):

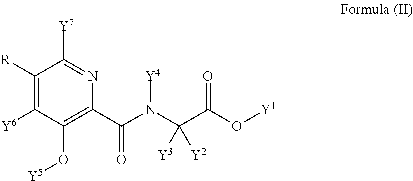

or a pharmaceutically acceptable salt, solvate or hydrate thereof, wherein

R is selected from:
(i) Y⁸; or
(ii) substituted or unsubstituted phenyl;

said substitution selected from:
(i) C(Y⁹⁻¹¹)₃;
(ii) halogen;
(iii) cyano;
(iv) Y¹²;
(v) Y¹³;
(vi) Y¹⁴;
(vii) Y¹⁵; or
(viii) Y¹⁶;

wherein one or more Y atoms (i.e., Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and/or Y¹⁶) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and Y¹⁶ are isotopically enriched with deuterium.

In certain more specific embodiments, all of Y¹, Y⁴, and Y⁵ are hydrogen.

In certain embodiments, one or more Y atoms of a compound of Formula (II) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 1

| | |
|---|---|
| 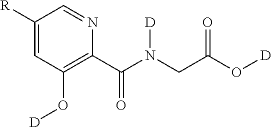 | 1 |
| 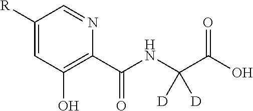 | 2 |
| 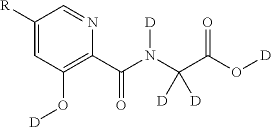 | 3 |
| 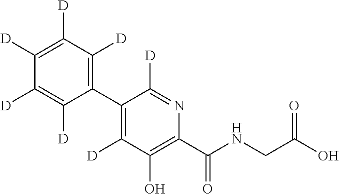 [SMILES: [2H]c1nc(C(=O)NCC(=O)O)c(O)c([2H])c1-c1c([2H])c([2H])c([2H])c([2H])c1[2H]] | 4 |
| 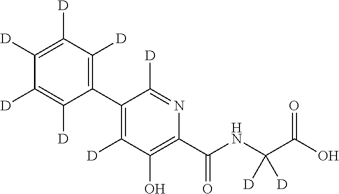 [SMILES: [2H]c1nc(C(=O)NC([2H])([2H])C(=O)O)c(O)c([2H])c1-c1c([2H])c([2H])c([2H])c([2H])c1[2H]] | 5 |
| 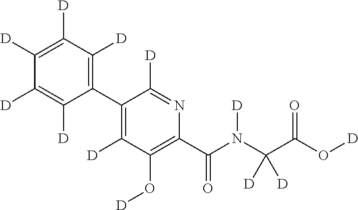 | 6 |
| 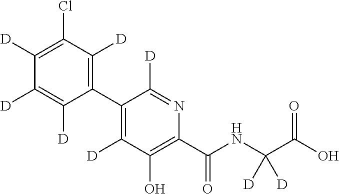 [SMILES: [2H]c1nc(C(=O)NC([2H])([2H])C(=O)O)c(O)c([2H])c1-c1c([2H])c([2H])c([2H])c(Cl)c1[2H]] | 7 |
| 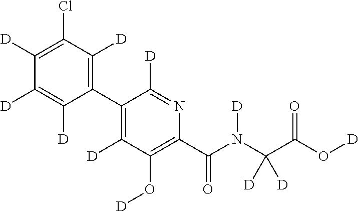 | 8 |
| 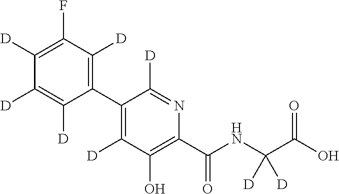 [SMILES: [2H]c1nc(C(=O)NC([2H])([2H])C(=O)O)c(O)c([2H])c1-c1c([2H])c([2H])c([2H])c(F)c1[2H]] | 9 |
| 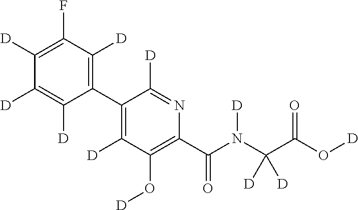 | 10 |
| 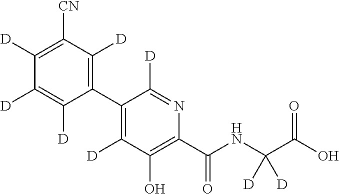 [SMILES: [2H]c1nc(C(=O)NC([2H])([2H])C(=O)O)c(O)c([2H])c1-c1c([2H])c([2H])c([2H])c(C#N)c1[2H]] | 11 |
| 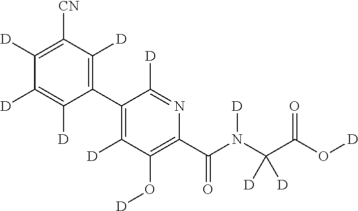 | 12 |
| 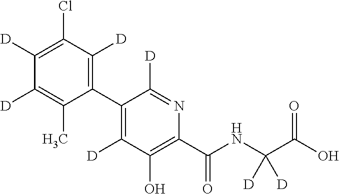 [SMILES: [2H]c1nc(C(=O)NC([2H])([2H])C(=O)O)c(O)c([2H])c1-c1c([2H])c(Cl)c([2H])c([2H])c1C] | 13 |
| 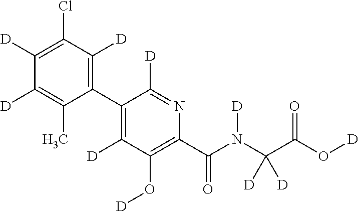 | 14 |
| 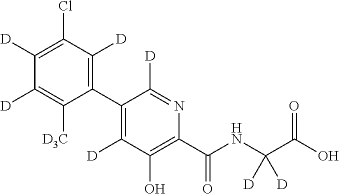 [SMILES: [2H]c1nc(C(=O)NC([2H])([2H])C(=O)O)c(O)c([2H])c1-c1c([2H])c(Cl)c([2H])c([2H])c1C([2H])([2H])[2H]] | 15 |
| 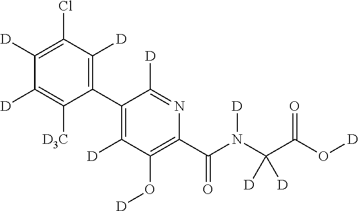 | 16 |

In certain more specific embodiments, the deuterium-enriched HIF prolyl hydroxylase inhibitor or HIF-alpha stabilizer has a structure of Formula (III):

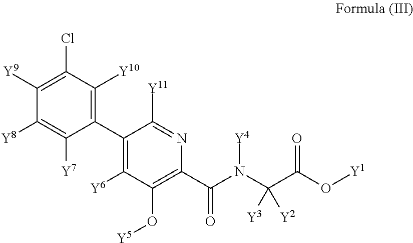

or a pharmaceutically acceptable salt, solvate, hydrate, or stereoisomer thereof,

wherein one or more Y atoms (i.e., Y¹, Y², Y³, Y⁴, Y⁵, Y⁶, Y⁷, Y⁸, Y⁹, Y¹⁰, and/or Y¹¹) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y³, Y⁴, Y⁵, Y⁶, Y⁷, Y⁸, Y⁹, Y¹⁰, and Y¹¹ are isotopically enriched with deuterium.

In certain more specific embodiments, all of Y¹, Y⁴, and Y⁵ are hydrogen.

In certain embodiments, one or more Y atoms on the phenyl portion of a compound of Formula (III) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 2

| | |
|---|---|
| 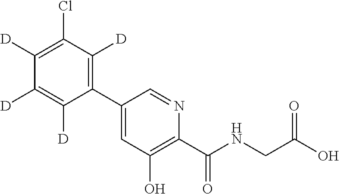 [SMILES: [2H]c1c([2H])c(Cl)c([2H])c(-c2cnc(C(=O)NCC(=O)O)c(O)c2)c1[2H]] | 17 |
| 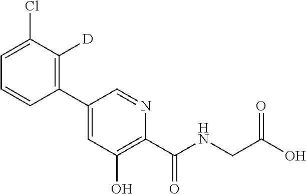 [SMILES: [2H]c1c(Cl)cccc1-c1cnc(C(=O)NCC(=O)O)c(O)c1] | 18 |
| 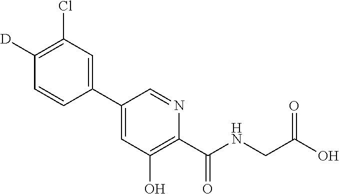 [SMILES: [2H]c1ccc(-c2cnc(C(=O)NCC(=O)O)c(O)c2)cc1Cl] | 19 |
| 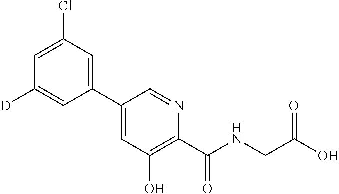 [SMILES: [2H]c1cc(Cl)cc(-c2cnc(C(=O)NCC(=O)O)c(O)c2)c1] | 20 |
| 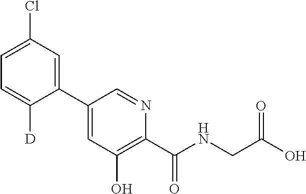 [SMILES: [2H]c1ccc(Cl)cc1-c1cnc(C(=O)NCC(=O)O)c(O)c1] | 21 |
| 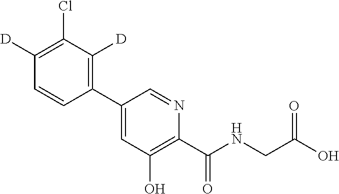 [SMILES: [2H]c1ccc(-c2cnc(C(=O)NCC(=O)O)c(O)c2)c([2H])c1Cl] | 22 |
| 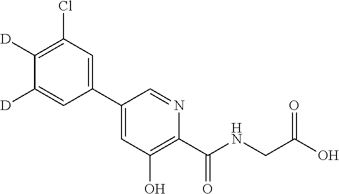 [SMILES: [2H]c1cc(-c2cnc(C(=O)NCC(=O)O)c(O)c2)cc(Cl)c1[2H]] | 23 |
| 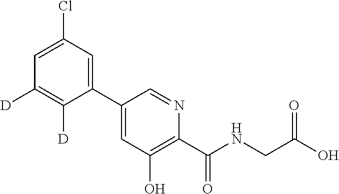 [SMILES: [2H]c1cc(Cl)cc(-c2cnc(C(=O)NCC(=O)O)c(O)c2)c1[2H]] | 24 |
| 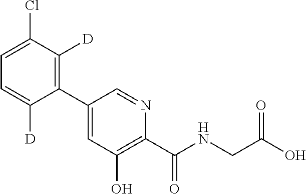 [SMILES: [2H]c1ccc(Cl)c([2H])c1-c1cnc(C(=O)NCC(=O)O)c(O)c1] | 25 |
| 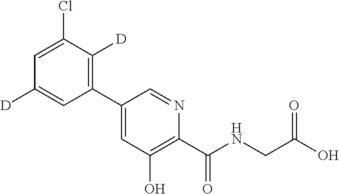 [SMILES: [2H]c1cc(Cl)c([2H])c(-c2cnc(C(=O)NCC(=O)O)c(O)c2)c1] | 26 |
| 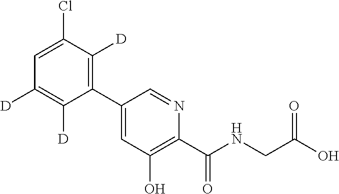 [SMILES: [2H]c1cc(Cl)c([2H])c(-c2cnc(C(=O)NCC(=O)O)c(O)c2)c1[2H]] | 27 |
| 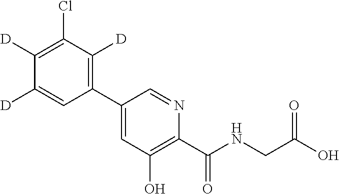 [SMILES: [2H]c1cc(-c2cnc(C(=O)NCC(=O)O)c(O)c2)c([2H])c(Cl)c1[2H]] | 28 |

In certain embodiments, one or more Y atoms on the pyridine portion of a compound of Formula (III) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 3

| | |
|---|---|
| 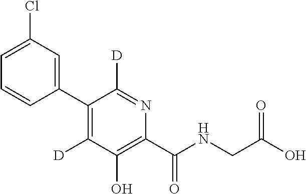 [SMILES: [2H]c1nc(C(=O)NCC(=O)O)c(O)c([2H])c1-c1cccc(Cl)c1] | 29 |
| 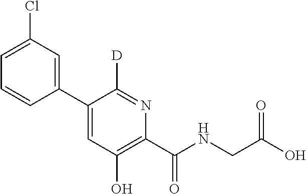 [SMILES: [2H]c1nc(C(=O)NCC(=O)O)c(O)cc1-c1cccc(Cl)c1] | 30 |
| 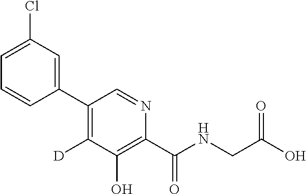 [SMILES: [2H]c1c(-c2cccc(Cl)c2)cnc(C(=O)NCC(=O)O)c1O] | 31 |

In certain embodiments, one or more Y atoms on the alkyl portion of a compound of Formula (III) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 4

| | |
|---|---|
| 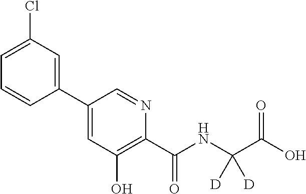 [SMILES: [2H]C([2H])(NC(=O)c1ncc(-c2cccc(Cl)c2)cc1O)C(=O)O] | 32 |
| 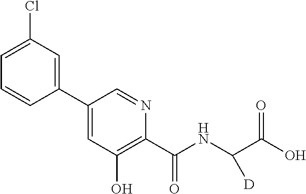 [SMILES: [2H]C(NC(=O)c1ncc(-c2cccc(Cl)c2)cc1O)C(=O)O] | 33 |

In certain embodiments, one or more Y atoms on the heteroatoms of a compound of Formula (III) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 5

| | |
|---|---|
| 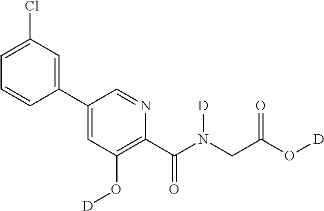 | 34 |
| 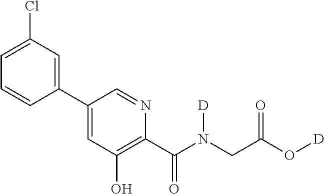 | 35 |
| 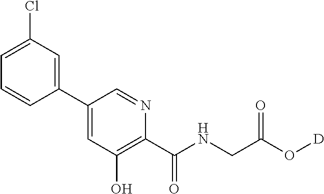 | 36 |
| 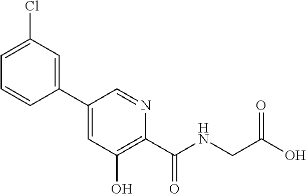 [SMILES: O=C(O)CNC(=O)c1ncc(-c2cccc(Cl)c2)cc1O] | 37 |
| 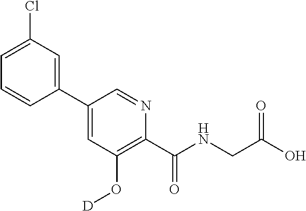 | 38 |
| 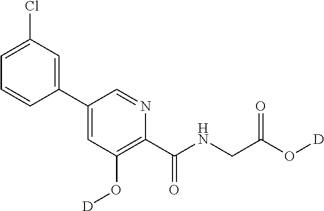 | 39 |
| 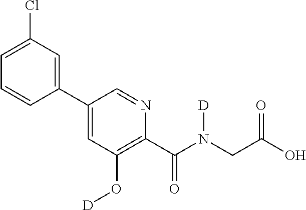 | 40 |

In certain embodiments, one or more Y atoms on the phenyl, pyridine, heteroatoms, and/or alkyl portions of a compound of Formula (III) is/are deuterium-enriched, i.e., any combination of deuterium-enrichment shown above is encompassed. In some embodiments the compound is selected from:

TABLE 6

| | |
|---|---|
| 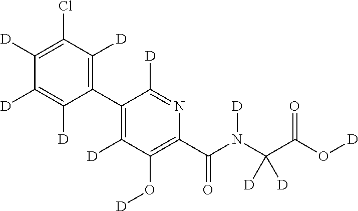 | 41 |
| 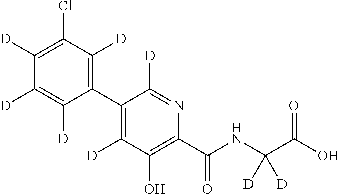 [SMILES: [2H]c1nc(C(=O)NC([2H])([2H])C(=O)O)c(O)c([2H])c1-c1c([2H])c([2H])c([2H])c(Cl)c1[2H]] | 42 |
| 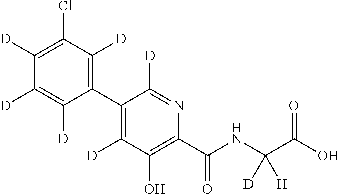 [SMILES: [2H]c1nc(C(=O)NC([2H])C(=O)O)c(O)c([2H])c1-c1c([2H])c([2H])c([2H])c(Cl)c1[2H]] | 43 |
| 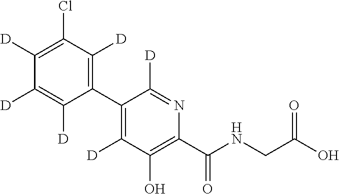 [SMILES: [2H]c1nc(C(=O)NCC(=O)O)c(O)c([2H])c1-c1c([2H])c([2H])c([2H])c(Cl)c1[2H]] | 44 |
| 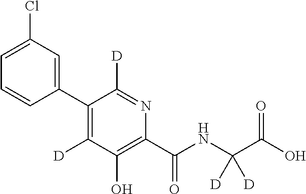 [SMILES: [2H]c1nc(C(=O)NC([2H])([2H])C(=O)O)c(O)c([2H])c1-c1cccc(Cl)c1] | 45 |
| 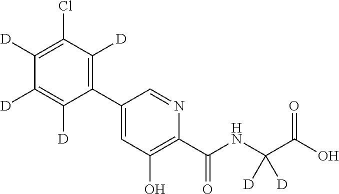 [SMILES: [2H]c1c([2H])c(Cl)c([2H])c(-c2cnc(C(=O)NC([2H])([2H])C(=O)O)c(O)c2)c1[2H]] | 46 |
| 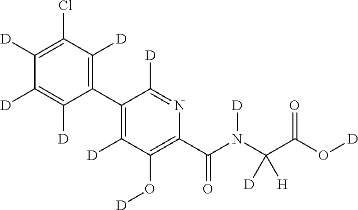 | 47 |
| 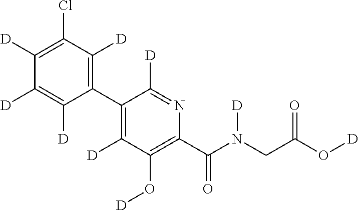 | 48 |
| 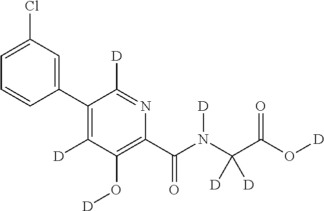 | 49 |
| 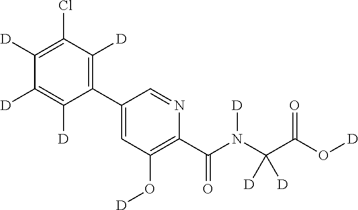 | 50 |

In certain more specific embodiments, the deuterium-enriched HIF prolyl hydroxylase inhibitor or HIF-alpha stabilizer has a structure of Formula (IV):

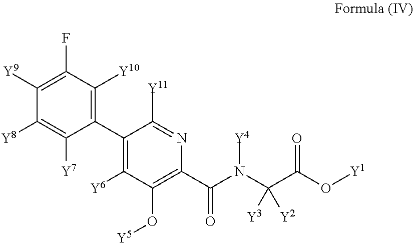

or a pharmaceutically acceptable salt, solvate, hydrate, or stereoisomer thereof,

wherein one or more Y atoms (i.e., Y¹, Y², Y³, Y⁴, Y⁵, Y⁶, Y⁷, Y⁸, Y⁹, Y¹⁰, and/or Y¹¹) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y³, Y⁴, Y⁵, Y⁶, Y⁷, Y⁸, Y⁹, Y¹⁰, and Y¹¹ are isotopically enriched with deuterium.

In certain more specific embodiments, all of Y¹, Y⁴, and Y⁵ are hydrogen.

In certain embodiments, one or more Y atoms on the phenyl portion of a compound of Formula (IV) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 7

| | |
|---|---|
| 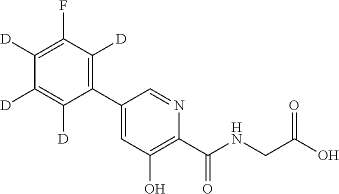 [SMILES: [2H]c1c([2H])c(F)c([2H])c(-c2cnc(C(=O)NCC(=O)O)c(O)c2)c1[2H]] | 51 |
| 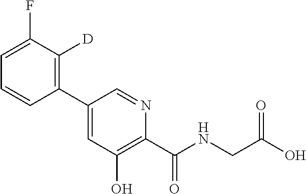 [SMILES: [2H]c1c(F)cccc1-c1cnc(C(=O)NCC(=O)O)c(O)c1] | 52 |
| 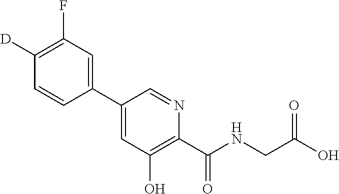 [SMILES: [2H]c1ccc(-c2cnc(C(=O)NCC(=O)O)c(O)c2)cc1F] | 53 |
| 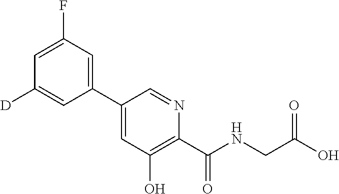 [SMILES: [2H]c1cc(F)cc(-c2cnc(C(=O)NCC(=O)O)c(O)c2)c1] | 54 |
| 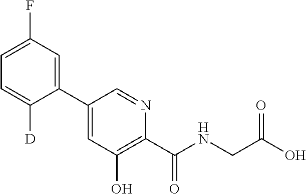 [SMILES: [2H]c1ccc(F)cc1-c1cnc(C(=O)NCC(=O)O)c(O)c1] | 55 |
| 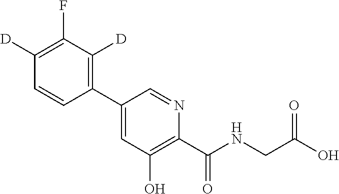 [SMILES: [2H]c1ccc(-c2cnc(C(=O)NCC(=O)O)c(O)c2)c([2H])c1F] | 56 |
| 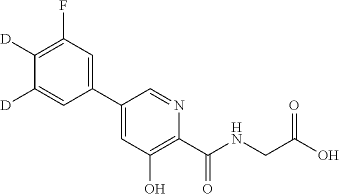 [SMILES: [2H]c1cc(-c2cnc(C(=O)NCC(=O)O)c(O)c2)cc(F)c1[2H]] | 57 |
| 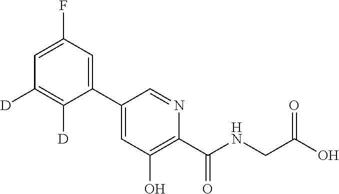 [SMILES: [2H]c1cc(F)cc(-c2cnc(C(=O)NCC(=O)O)c(O)c2)c1[2H]] | 58 |
| 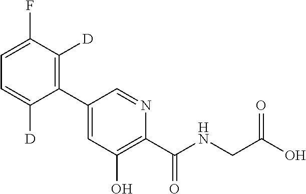 [SMILES: [2H]c1ccc(F)c([2H])c1-c1cnc(C(=O)NCC(=O)O)c(O)c1] | 59 |
| 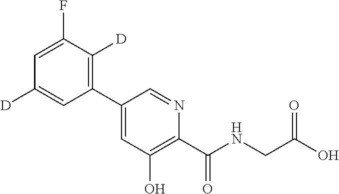 [SMILES: [2H]c1cc(F)c([2H])c(-c2cnc(C(=O)NCC(=O)O)c(O)c2)c1] | 60 |
| 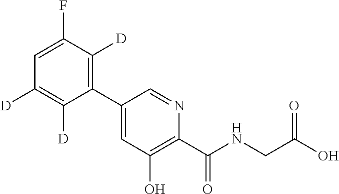 [SMILES: [2H]c1cc(F)c([2H])c(-c2cnc(C(=O)NCC(=O)O)c(O)c2)c1[2H]] | 61 |
| 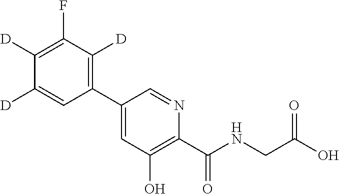 [SMILES: [2H]c1cc(-c2cnc(C(=O)NCC(=O)O)c(O)c2)c([2H])c(F)c1[2H]] | 62 |

In certain embodiments, one or more Y atoms on the pyridine portion of a compound of Formula (IV) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 8

| | |
|---|---|
| 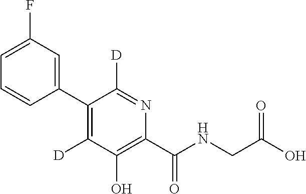 [SMILES: [2H]c1nc(C(=O)NCC(=O)O)c(O)c([2H])c1-c1cccc(F)c1] | 63 |
| 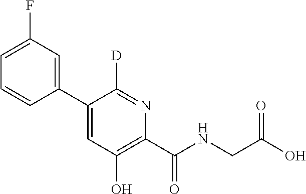 [SMILES: [2H]c1nc(C(=O)NCC(=O)O)c(O)cc1-c1cccc(F)c1] | 64 |
| 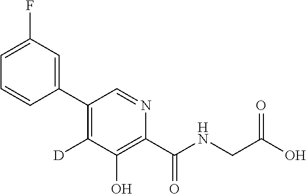 [SMILES: [2H]c1c(-c2cccc(F)c2)cnc(C(=O)NCC(=O)O)c1O] | 65 |

In certain embodiments, one or more Y atoms on the alkyl portion of a compound of Formula (IV) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 9

| | |
|---|---|
| 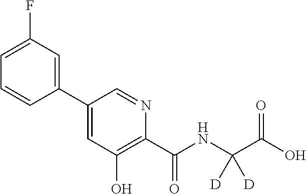 [SMILES: [2H]C([2H])(NC(=O)c1ncc(-c2cccc(F)c2)cc1O)C(=O)O] | 66 |
| 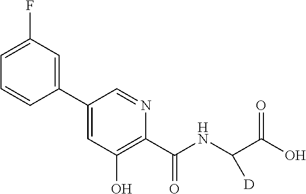 [SMILES: [2H]C(NC(=O)c1ncc(-c2cccc(F)c2)cc1O)C(=O)O] | 67 |

In certain embodiments, one or more Y atoms on the heteroatoms of a compound of Formula (IV) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 10

| | |
|---|---|
| 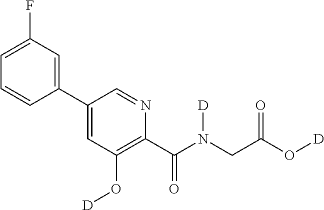 | 68 |
| 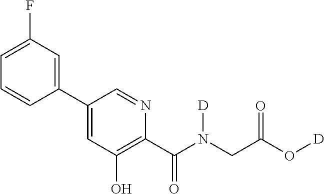 | 69 |
| 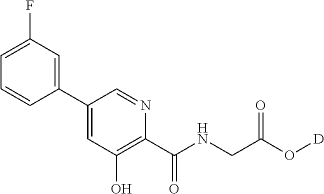 | 70 |
| 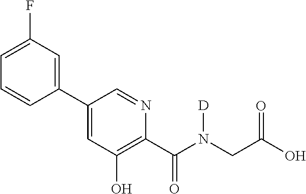 | 71 |
| 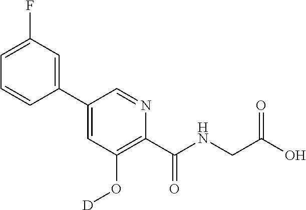 | 72 |
| 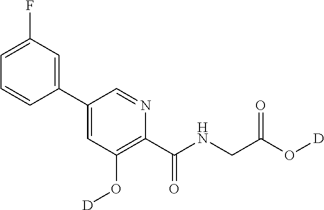 | 73 |
| 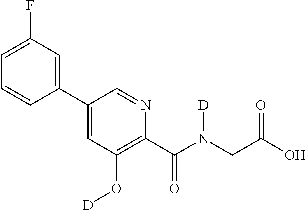 | 74 |

In certain embodiments, one or more Y atoms on the phenyl, pyridine, heteroatoms, and/or alkyl portions of a compound of Formula (IV) is/are deuterium-enriched, i.e., any combination of deuterium-enrichment shown above is encompassed. In some embodiments the compound is selected from:

TABLE 11

| | |
|---|---|
| 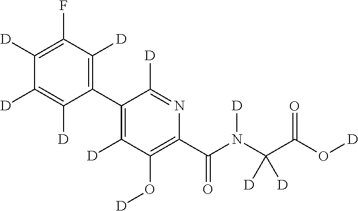 | 75 |
| 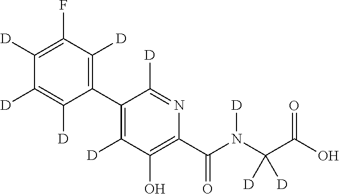 | 76 |
| 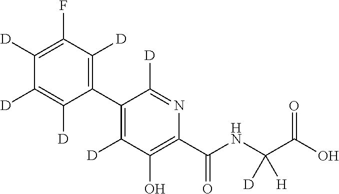 [SMILES: [2H]c1nc(C(=O)NC([2H])C(=O)O)c(O)c([2H])c1-c1c([2H])c([2H])c([2H])c(F)c1[2H]] | 77 |
| 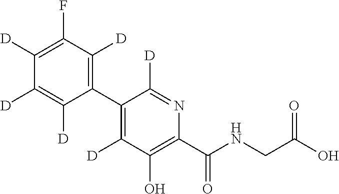 [SMILES: [2H]c1nc(C(=O)NCC(=O)O)c(O)c([2H])c1-c1c([2H])c([2H])c([2H])c(F)c1[2H]] | 78 |
| 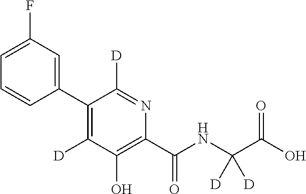 [SMILES: [2H]c1nc(C(=O)NC([2H])([2H])C(=O)O)c(O)c([2H])c1-c1cccc(F)c1] | 79 |
| 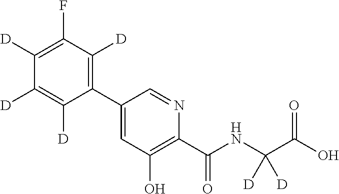 [SMILES: [2H]c1c([2H])c(F)c([2H])c(-c2cnc(C(=O)NC([2H])([2H])C(=O)O)c(O)c2)c1[2H]] | 80 |
| 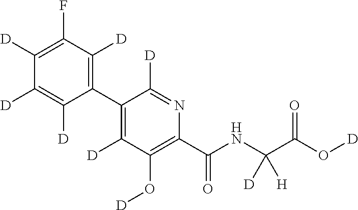 | 81 |
| 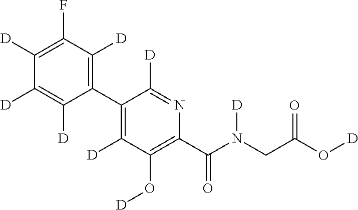 | 82 |
| 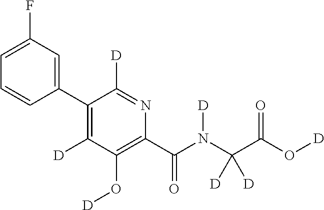 | 83 |
| 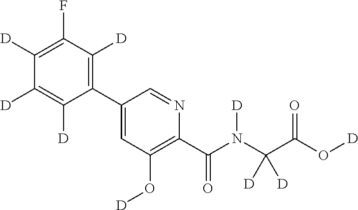 | 84 |

In certain more specific embodiments, the deuterium-enriched HIF prolyl hydroxylase inhibitor or HIF-alpha stabilizer has a structure of Formula (V):

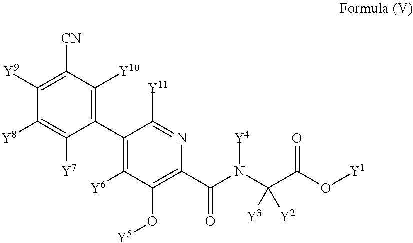

or a pharmaceutically acceptable salt, solvate, hydrate, or stereoisomer thereof,

wherein one or more Y atoms (i.e., Y¹, Y², Y³, Y⁴, Y⁵, Y⁶, Y⁷, Y⁸, Y⁹, Y¹⁰, and/or Y¹¹) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y³, Y⁴, Y⁵, Y⁶, Y⁷, Y⁸, Y⁹, Y¹⁰, and Y¹¹ are isotopically enriched with deuterium.

In certain more specific embodiments, all of Y¹, Y⁴, and Y⁵ are hydrogen.

In certain embodiments, one or more Y atoms on the phenyl portion of a compound of Formula (V) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 12

| | |
|---|---|
| 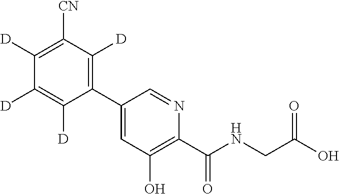 [SMILES: [2H]c1c([2H])c(C#N)c([2H])c(-c2cnc(C(=O)NCC(=O)O)c(O)c2)c1[2H]] | 85 |
| 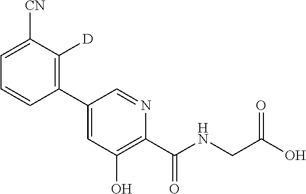 [SMILES: [2H]c1c(C#N)cccc1-c1cnc(C(=O)NCC(=O)O)c(O)c1] | 86 |
| 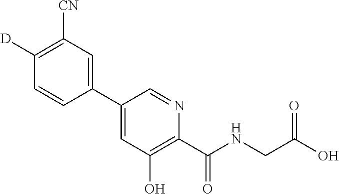 [SMILES: [2H]c1ccc(-c2cnc(C(=O)NCC(=O)O)c(O)c2)cc1C#N] | 87 |
| 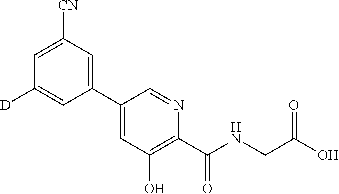 [SMILES: [2H]c1cc(C#N)cc(-c2cnc(C(=O)NCC(=O)O)c(O)c2)c1] | 88 |
| 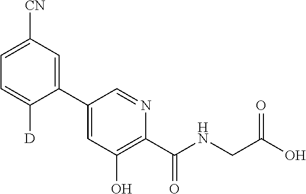 [SMILES: [2H]c1ccc(C#N)cc1-c1cnc(C(=O)NCC(=O)O)c(O)c1] | 89 |
| 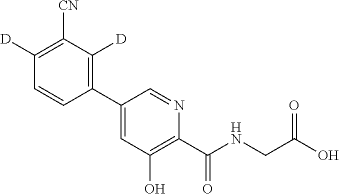 [SMILES: [2H]c1ccc(-c2cnc(C(=O)NCC(=O)O)c(O)c2)c([2H])c1C#N] | 90 |
| 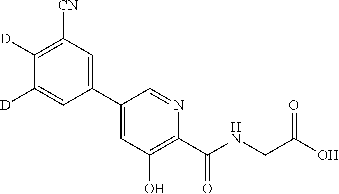 [SMILES: [2H]c1cc(-c2cnc(C(=O)NCC(=O)O)c(O)c2)cc(C#N)c1[2H]] | 91 |
| 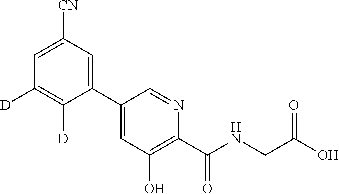 [SMILES: [2H]c1cc(C#N)cc(-c2cnc(C(=O)NCC(=O)O)c(O)c2)c1[2H]] | 92 |
| 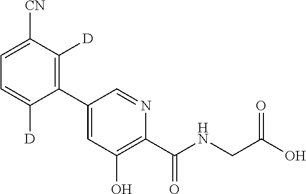 [SMILES: [2H]c1ccc(C#N)c([2H])c1-c1cnc(C(=O)NCC(=O)O)c(O)c1] | 93 |
| 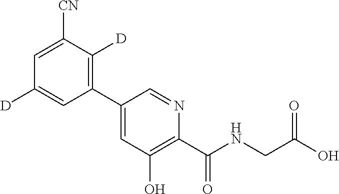 [SMILES: [2H]c1cc(C#N)c([2H])c(-c2cnc(C(=O)NCC(=O)O)c(O)c2)c1] | 94 |
| 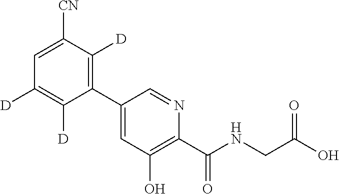 [SMILES: [2H]c1cc(C#N)c([2H])c(-c2cnc(C(=O)NCC(=O)O)c(O)c2)c1[2H]] | 95 |
| 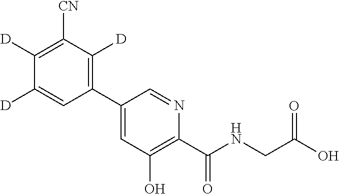 [SMILES: [2H]c1cc(-c2cnc(C(=O)NCC(=O)O)c(O)c2)c([2H])c(C#N)c1[2H]] | 96 |

In certain embodiments, one or more Y atoms on the pyridine portion of a compound of Formula (V) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 13

| | |
|---|---|
| 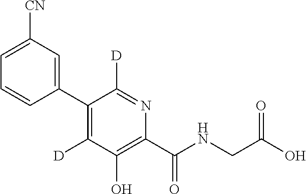 [SMILES: [2H]c1nc(C(=O)NCC(=O)O)c(O)c([2H])c1-c1cccc(C#N)c1] | 97 |
| 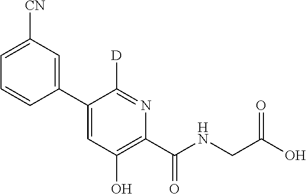 [SMILES: [2H]c1nc(C(=O)NCC(=O)O)c(O)cc1-c1cccc(C#N)c1] | 98 |
| 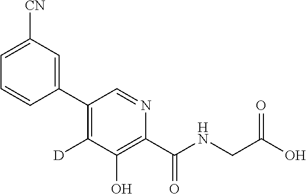 [SMILES: [2H]c1c(-c2cccc(C#N)c2)cnc(C(=O)NCC(=O)O)c1O] | 99 |

In certain embodiments, one or more Y atoms on the alkyl portion of a compound of Formula (V) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 14

| | |
|---|---|
| 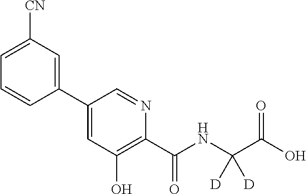 [SMILES: [2H]C([2H])(NC(=O)c1ncc(-c2cccc(C#N)c2)cc1O)C(=O)O] | 100 |
| 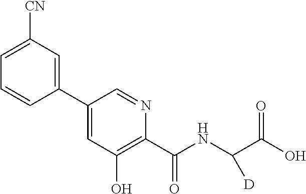 [SMILES: [2H]C(NC(=O)c1ncc(-c2cccc(C#N)c2)cc1O)C(=O)O] | 101 |

In certain embodiments, one or more Y atoms on the heteroatoms of a compound of Formula (V) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 15

| | |
|---|---|
| 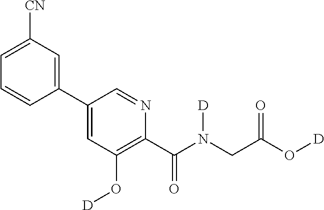 | 102 |
| 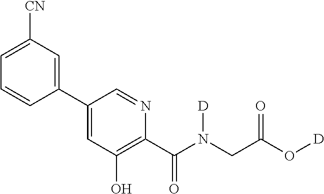 | 103 |
| 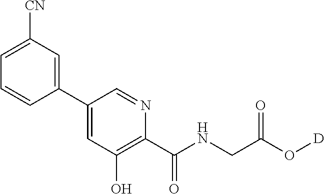 | 104 |
| 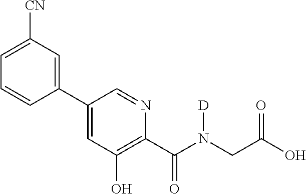 | 105 |
| 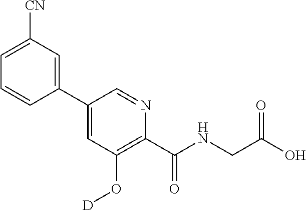 | 106 |
| 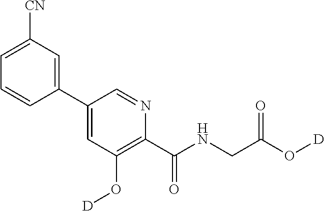 | 107 |
| 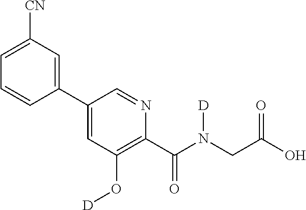 | 108 |

In certain embodiments, one or more Y atoms on the phenyl, pyridine, heteroatoms, and/or alkyl portions of a compound of Formula (V) is/are deuterium-enriched, i.e., any combination of deuterium-enrichment shown above is encompassed. In some embodiments the compound is selected from:

TABLE 16

| | |
|---|---|
| 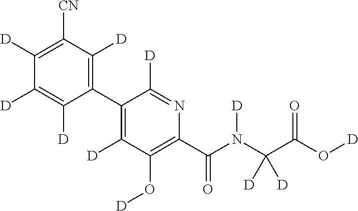 | 109 |
| 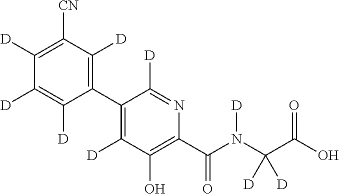 | 110 |
| 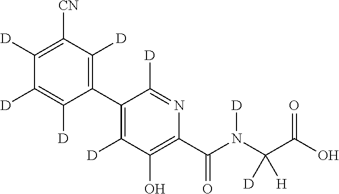 | 111 |
| 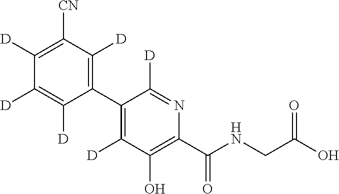 [SMILES: [2H]c1nc(C(=O)NCC(=O)O)c(O)c([2H])c1-c1c([2H])c([2H])c([2H])c(C#N)c1[2H]] | 112 |
| 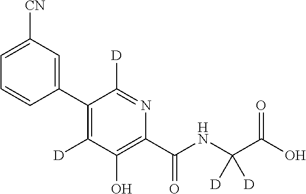 [SMILES: [2H]c1nc(C(=O)NC([2H])([2H])C(=O)O)c(O)c([2H])c1-c1cccc(C#N)c1] | 113 |
| 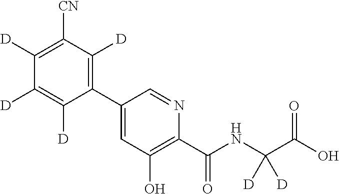 [SMILES: [2H]c1c([2H])c(C#N)c([2H])c(-c2cnc(C(=O)NC([2H])([2H])C(=O)O)c(O)c2)c1[2H]] | 114 |
| 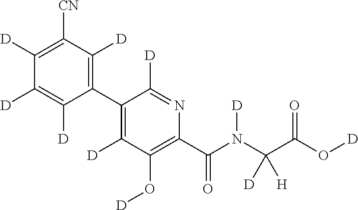 | 115 |
| 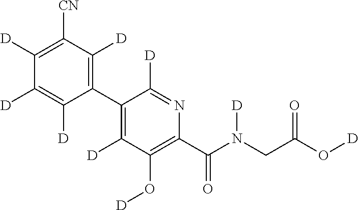 | 116 |
| 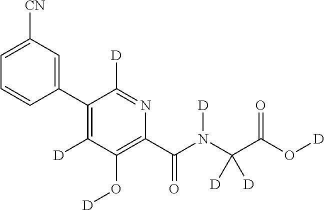 | 117 |
| 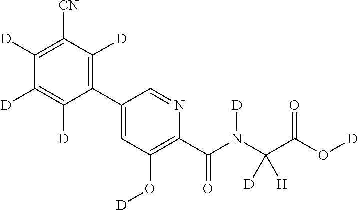 | 118 |

In certain more specific embodiments, the deuterium-enriched HIF prolyl hydroxylase inhibitor or HIF-alpha stabilizer has a structure of Formula (VI):

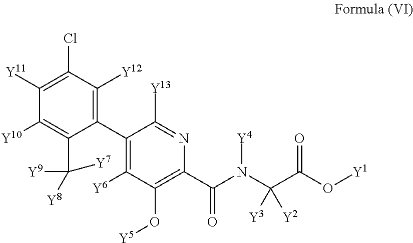

or a pharmaceutically acceptable salt, solvate, hydrate, or stereoisomer thereof,

wherein one or more Y atoms (i.e., Y¹, Y², Y³, Y⁴, Y⁵, Y⁶, Y⁷, Y⁸, Y⁹, Y¹⁰, Y¹¹, Y¹², and/or Y¹³) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y³, Y⁴, Y⁵, Y⁶, Y⁷, Y⁸, Y⁹, Y¹⁰, Y¹¹, Y¹², and Y¹³ are isotopically enriched with deuterium.

In certain more specific embodiments, all of Y¹, Y⁴, and Y⁵ are hydrogen.

In certain embodiments, one or more Y atoms on the phenyl portion of a compound of Formula (VI) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 17

| | |
|---|---|
| 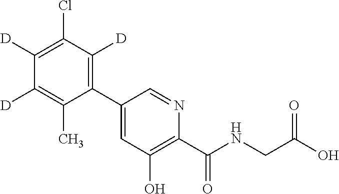 [SMILES: [2H]c1c([2H])c(Cl)c([2H])c(-c2cnc(C(=O)NCC(=O)O)c(O)c2)c1C] | 119 |
| 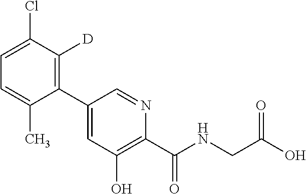 [SMILES: [2H]c1c(Cl)ccc(C)c1-c1cnc(C(=O)NCC(=O)O)c(O)c1] | 120 |
| 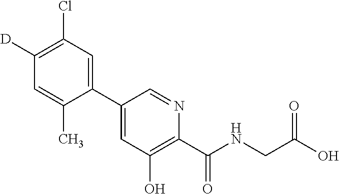 [SMILES: [2H]c1cc(C)c(-c2cnc(C(=O)NCC(=O)O)c(O)c2)cc1Cl] | 121 |
| 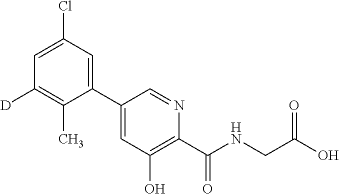 [SMILES: [2H]c1cc(Cl)cc(-c2cnc(C(=O)NCC(=O)O)c(O)c2)c1C] | 122 |
| 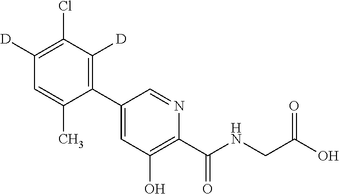 [SMILES: [2H]c1cc(C)c(-c2cnc(C(=O)NCC(=O)O)c(O)c2)c([2H])c1Cl] | 123 |
| 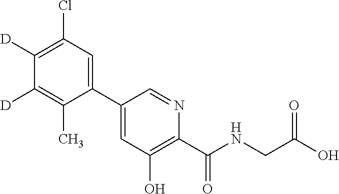 [SMILES: [2H]c1c(Cl)cc(-c2cnc(C(=O)NCC(=O)O)c(O)c2)c(C)c1[2H]] | 124 |
| 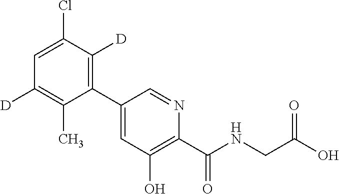 [SMILES: [2H]c1cc(Cl)c([2H])c(-c2cnc(C(=O)NCC(=O)O)c(O)c2)c1C] | 125 |

In certain embodiments, one or more Y atoms on the pyridine portion of a compound of Formula (VI) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 18

| | |
|---|---|
| 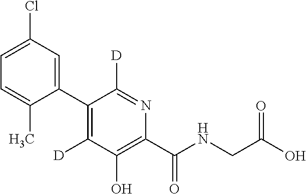 [SMILES: [2H]c1nc(C(=O)NCC(=O)O)c(O)c([2H])c1-c1cc(Cl)ccc1C] | 126 |
| 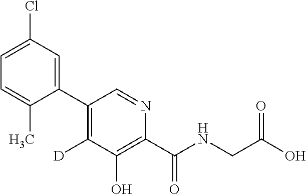 [SMILES: [2H]c1c(-c2cc(Cl)ccc2C)cnc(C(=O)NCC(=O)O)c1O] | 127 |
| 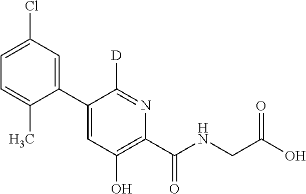 [SMILES: [2H]c1nc(C(=O)NCC(=O)O)c(O)cc1-c1cc(Cl)ccc1C] | 128 |

In certain embodiments, one or more Y atoms on the alkyl portion of a compound of Formula (VI) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 19

| | |
|---|---|
| 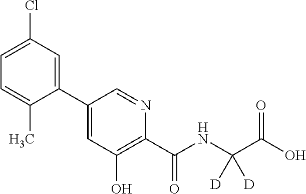 [SMILES: [2H]C([2H])(NC(=O)c1ncc(-c2cc(Cl)ccc2C)cc1O)C(=O)O] | 129 |
| 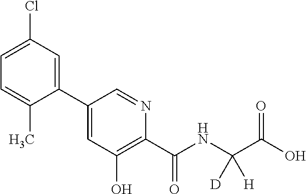 [SMILES: [2H]C(NC(=O)c1ncc(-c2cc(Cl)ccc2C)cc1O)C(=O)O] | 130 |

In certain embodiments, one or more Y atoms on the tert-butyl portion of a compound of Formula (VI) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 20

| | |
|---|---|
| 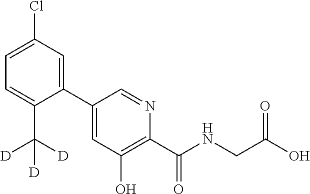 [SMILES: [2H]C([2H])([2H])c1ccc(Cl)cc1-c1cnc(C(=O)NCC(=O)O)c(O)c1] | 131 |
| 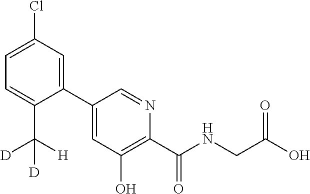 [SMILES: [2H]C([2H])c1ccc(Cl)cc1-c1cnc(C(=O)NCC(=O)O)c(O)c1] | 132 |
| 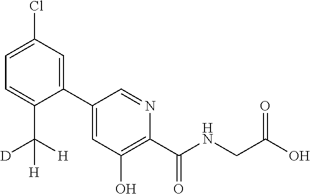 [SMILES: [2H]Cc1ccc(Cl)cc1-c1cnc(C(=O)NCC(=O)O)c(O)c1] | 133 |

In certain embodiments, one or more Y atoms on the heteroatoms of a compound of Formula (VI) is/are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium, and any atom not designated as a deuterium is present at its natural abundance:

TABLE 21

| | |
|---|---|
| 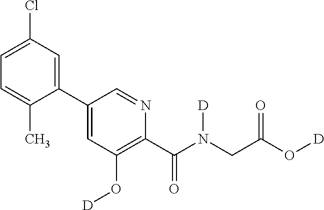 | 134 |
| 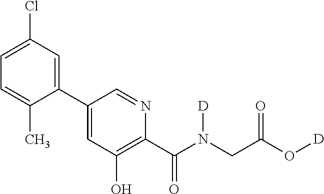 | 135 |
| 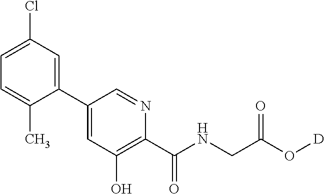 | 136 |
| 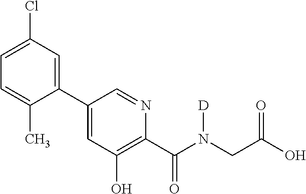 | 137 |
| 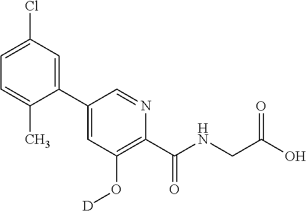 | 138 |
| 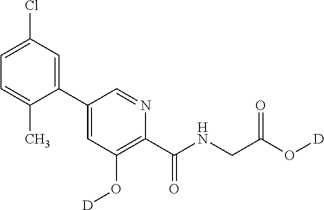 | 139 |
| 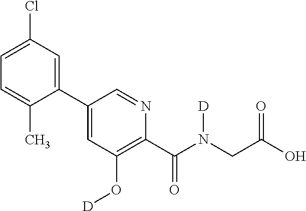 | 140 |

In certain embodiments, one or more Y atoms on the phenyl, pyridine, alkyl, heteroatoms, and/or tert-butyl portions of a compound of Formula (VI) is/are deuterium-enriched, i.e., any combination of deuterium-enrichment shown above is encompassed. In some embodiments the compound is selected from:

TABLE 22

| | |
|---|---|
| 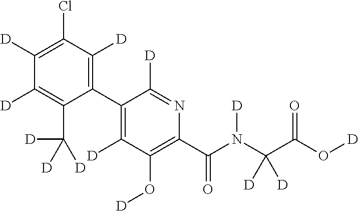 | 141 |
| 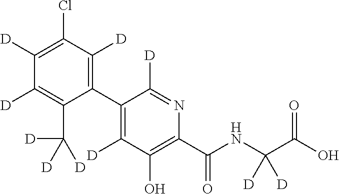 [SMILES: [2H]c1nc(C(=O)NC([2H])([2H])C(=O)O)c(O)c([2H])c1-c1c([2H])c(Cl)c([2H])c([2H])c1C([2H])([2H])[2H]] | 142 |
| 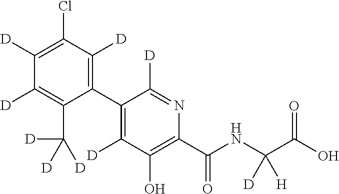 [SMILES: [2H]c1nc(C(=O)NC([2H])C(=O)O)c(O)c([2H])c1-c1c([2H])c(Cl)c([2H])c([2H])c1C([2H])([2H])[2H]] | 143 |
| 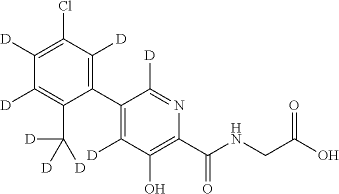 [SMILES: [2H]c1nc(C(=O)NCC(=O)O)c(O)c([2H])c1-c1c([2H])c(Cl)c([2H])c([2H])c1C([2H])([2H])[2H]] | 144 |
| 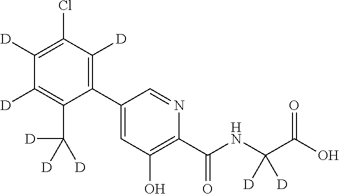 [SMILES: [2H]c1c([2H])c(C([2H])([2H])[2H])c(-c2cnc(C(=O)NC([2H])([2H])C(=O)O)c(O)c2)c([2H])c1Cl] | 145 |
| 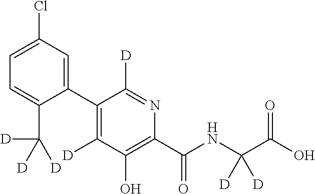 [SMILES: [2H]c1nc(C(=O)NC([2H])([2H])C(=O)O)c(O)c([2H])c1-c1cc(Cl)ccc1C([2H])([2H])[2H]] | 146 |
| 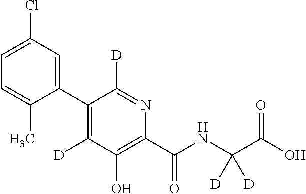 [SMILES: [2H]c1nc(C(=O)NC([2H])([2H])C(=O)O)c(O)c([2H])c1-c1cc(Cl)ccc1C] | 147 |
| 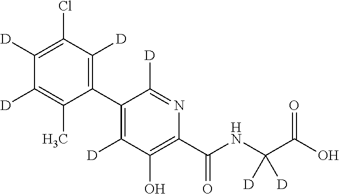 [SMILES: [2H]c1nc(C(=O)NC([2H])([2H])C(=O)O)c(O)c([2H])c1-c1c([2H])c(Cl)c([2H])c([2H])c1C] | 148 |
| 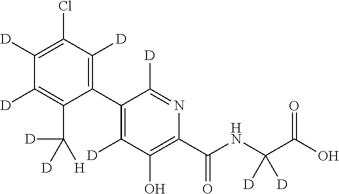 [SMILES: [2H]c1nc(C(=O)NC([2H])([2H])C(=O)O)c(O)c([2H])c1-c1c([2H])c(Cl)c([2H])c([2H])c1C([2H])[2H]] | 149 |
| 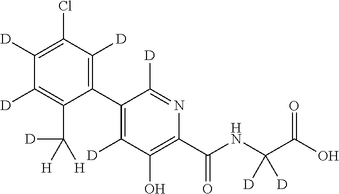 [SMILES: [2H]Cc1c([2H])c([2H])c(Cl)c([2H])c1-c1c([2H])nc(C(=O)NC([2H])([2H])C(=O)O)c(O)c1[2H]] | 150 |
| 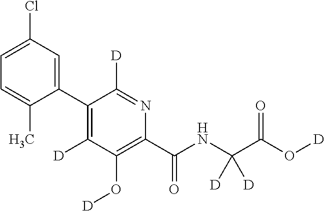 | 151 |
| 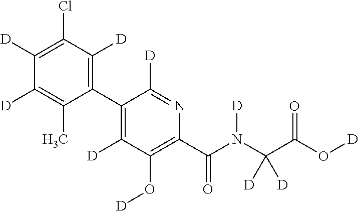 | 152 |
| 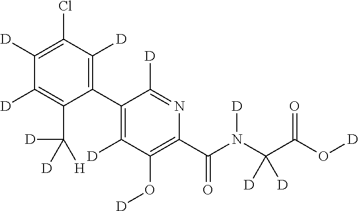 | 153 |
| 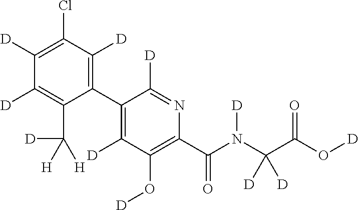 | 154 |

In certain embodiments, a metabolite of a compound has a structure of Formula (I), Formula (II), Formula (III), Formula (IV), Formula (V), or of Formula (VI). In certain more specific embodiments, such a metabolite is a phenolic glucuronide having the structure of Metabolite 1 or an acyl-glucuronide having a structure of Metabolite 2.

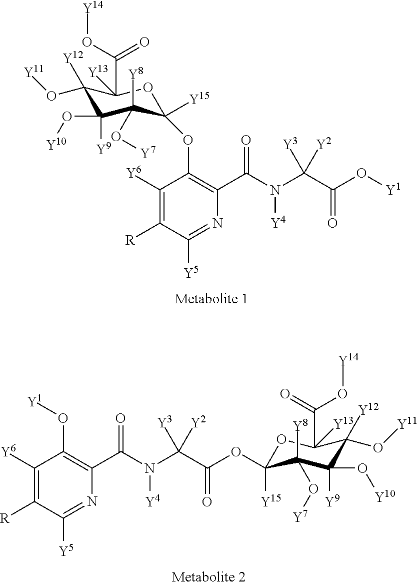

or a pharmaceutically acceptable salt, solvate, hydrate, or stereoisomer thereof, wherein

R is selected from:
(i) Y¹⁶; or
(ii) substituted or unsubstituted phenyl;

said substitution selected from:
(i) C(Y¹⁷⁻¹⁹)₃;
(ii) halogen;
(iii) cyano;
(iv) Y²⁰;
(v) Y²¹;
(vi) Y²²;
(vii) Y²³; or
(viii) Y²⁴;

wherein one or more Y atoms(i.e., Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, and/or Y²⁴) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, Y²⁰, Y²¹, Y²², Y²³, and Y²⁴ are isotopically enriched with deuterium.

In certain more specific embodiments, all of Y¹, Y⁴, Y⁷, Y¹⁰, Y¹¹, and Y¹⁴ are hydrogen.

In certain embodiments, a compound selected from Metabolite 1 or Metabolite 2 is isolated.

### 4.2.1 Synthesis

The compounds described herein may be synthesized using methods known to those of ordinary skill in the art. For example, particular compounds described herein are synthesized using standard synthetic organic chemistry techniques known to those of ordinary skill in the art.

In certain embodiments, known procedures for the synthesis of HIF prolyl hydroxylase enzyme inhibitors of the Formula (I), Formula (II), Formula (III), Formula (IV), Formula (V), or Formula (VI) or a compound selected from Metabolite 1 or Metabolite 2 are employed, wherein one or more of the reagents, starting materials, precursors, or intermediates are replaced by one or more deuterium-enriched reagents or intermediates. Such known procedures for the synthesis of HIF prolyl hydroxylase enzyme inhibitors include, but are not limited to, those described in U.S. Patent Application 2012/0309977, which is incorporated herein by reference in its entirety. Deuterium-enriched reagents, starting materials, precursors, and intermediates are commercially available or may be prepared by routine chemical reactions known to one of skill in the art.

Lanthier et al. (U.S. Patent Application 2012/0309977) described a procedure for synthesizing a compound of Formula (II) starting from 3-chloroboronic acid and 3,5-dichloropicolinonitrile, as shown in the scheme below:

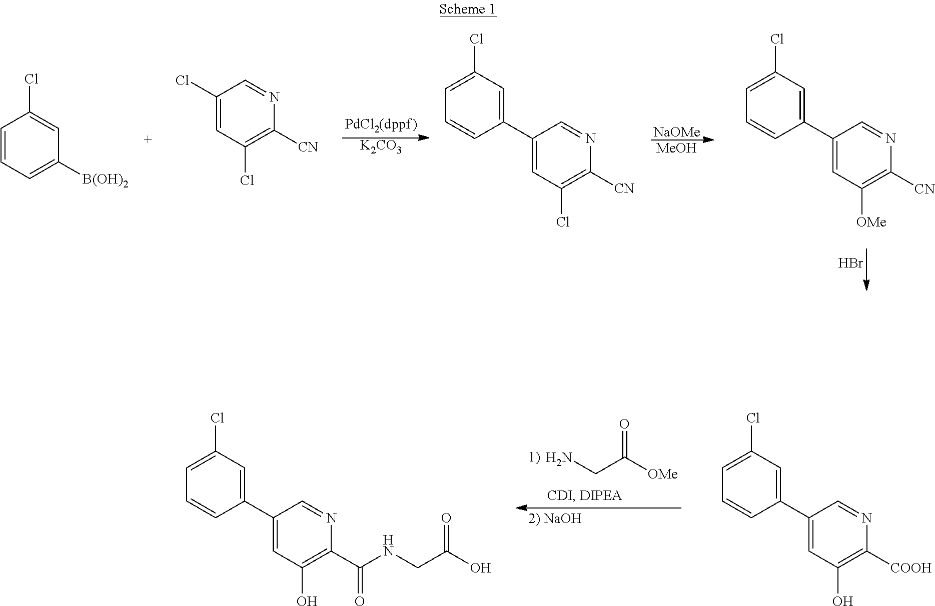

In certain embodiments, one or more hydrogen positions of the glycine methyl ester portion of a compound of Formula (II) are enriched with deuterium through organic synthesis. In some embodiments, the methods of Lanthier et al. are employed.

In certain embodiments, the methods of Lanthier et al. are employed, wherein a deuterium-enriched glycine methyl ester is used in the reaction, as shown in the scheme below:

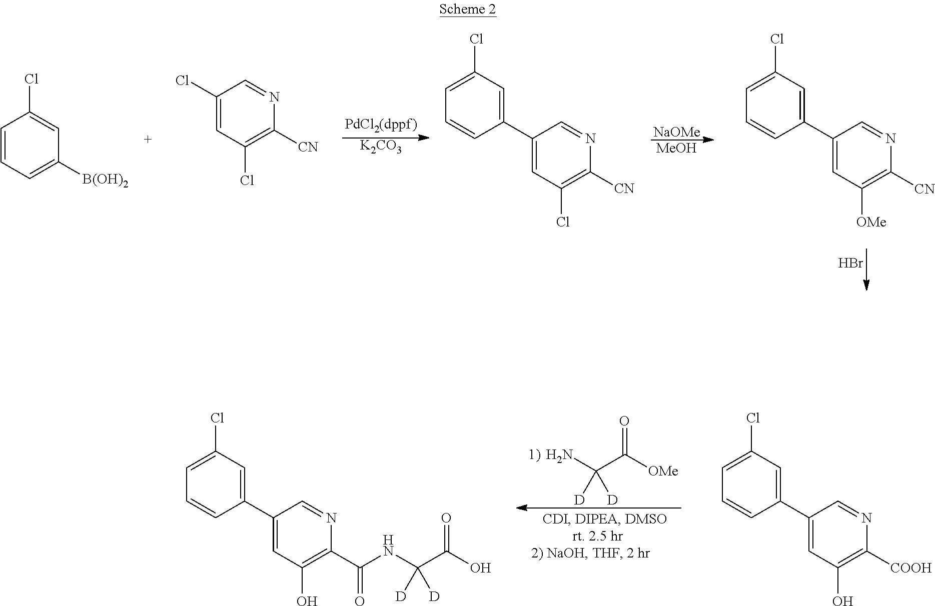

Deuterium-enriched glycine methyl ester may be obtained commercially or through techniques known to those of skill in the art.

In certain embodiments, one or more hydrogen sites of a compound of Formula (I), Formula (II), Formula (III), Formula (IV), Formula (V), or Formula (VI) or a compound selected from Metabolite 1 or Metabolite 2 are enriched with deuterium through organic synthesis as depicted in the following scheme:

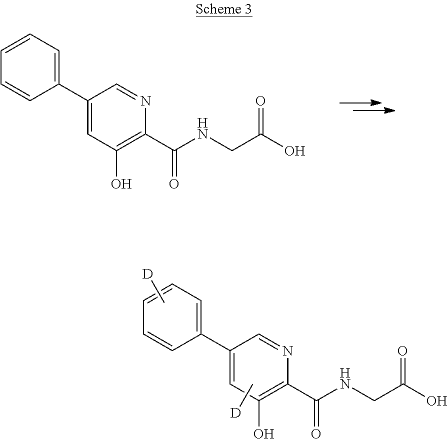

Such conditions are known to those of ordinary skill in the art including for example, those disclosed in the following references, each of which are incorporated herein by reference in their entireties: U.S. Publication No. 2007/0255076; U.S. Pat. No. 8,093,422; March, I. “Advanced Organic Chemistry, Reactions, Mechanisms, and Structure,” Sixth Ed., Wiley, New York, 2007; Larsen et al., J. Org. Chem., 1978, 43 (18), pp 3602-3602; Blake et al., J. Chem. Soc., Chem. Commun., 1975, 930; and references cited therein.

In certain embodiments, one or more hydrogen sites of a compound of Formula (I), Formula (II), Formula (III), Formula (IV), Formula (V), or Formula (VI) or a compound selected from Metabolite 1 or Metabolite 2 are enriched with deuterium through organic synthesis as depicted in the following scheme:

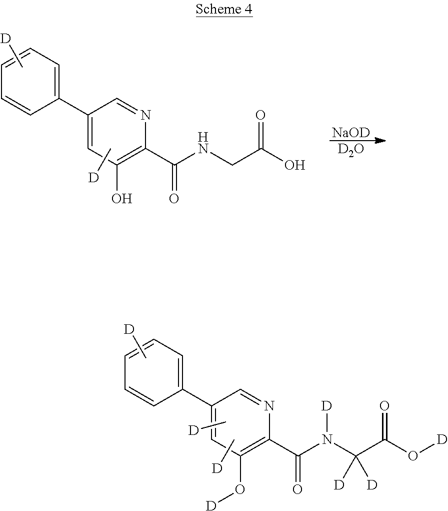

Such conditions are known to those of ordinary skill in the art including for example, those disclosed in the following references, each of which are incorporated herein by reference in their entireties: Atzrodt, J. et al. Angew. Chem. Int. Ed. 2007, 46, 7744; Wähälä, K. et al. J. Labelled Compd. Radiopharm. 1995, 36, 493; Rose, J. E. et al., J. Chem. Soc. Perkin Trans. 1995, 157; and references cited therein.

### 4.3 Therapeutic Applications

Provided herein are methods of using deuterium-enriched compounds to treat medical disorders. The deuterium-enriched compounds can be a compound of Formula (I), Formula (II), Formula (III), Formula (IV), Formula (V), or Formula (VI) or a compound selected from Metabolite 1 or Metabolite 2. The therapeutic methods comprise administering to a patient in need thereof a therapeutically effective amount of a deuterium-enriched compound described herein to treat the disorder.

In certain more specific embodiments, provided herein are methods of using the deuterium-enriched compounds of Formula (I), Formula (II), Formula (III), Formula (IV), Formula (V), or Formula (VI) or a compound selected from Metabolite 1 or Metabolite 2 or pharmaceutically acceptable salt, solvate, hydrate, or stereoisomer thereof for treating, preventing, and/or managing various diseases or disorders using a compound provided herein.

Without being limited by a particular theory, compounds provided herein can modulate hypoxia-inducible factor (HIF) prolyl hydroxylase, resulting in stabilization of HIFα (i.e., the degradation of HIFα is reduced). As a consequence of stabilizing HIFα, the transcription of various target genes is affected. Consequently, without being limited by a particular theory, some or all of such characteristics possessed by the compounds provided herein may render them useful in treating, managing, and/or preventing various diseases or disorders.

Examples of diseases or disorders include, but are not limited to, kidney disease and anemia.

In certain more specific embodiments, HIF stabilizers have been used for the treatment of cancer and are described in U.S. Patent Publication No. 2012/0329836, which is incorporated herein by reference in its entirety.

Examples of cancer and precancerous conditions include, but are not limited to, Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytoma/Malignant Glioma; Craniopharyngioma; Ependymoblastoma; Ependymoma; Medulloblastoma; Medulloepithelioma; Pineal Parenchymal Tumors of Intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma; Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T -Cell Lymphoma; Esophageal Cancer; Ewing Family of Tumors; Extra gonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; Intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T -Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, Intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T -Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; or Wilms Tumor.

Doses of a compound provided herein, or a pharmaceutically salt, solvate, hydrate, or stereoisomer thereof, vary depending on factors such as specific indication to be treated, prevented, or managed; and age and condition of a patient.

### 4.4 Assays

Without being limited by a particular theory, the deuterium-enriched compounds of a drug provided herein can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy.

Any assay known to the skilled artisan can be used to confirm the suitability of a compound provided herein for the methods provided herein, including enzyme-linked immunosorbent assay (ELISA), enzyme immunoassay (EIA), radio-immunoassay format (RIA), and/or surface plasmon resonance (SPR).

Additional analytical techniques can be used to confirm the suitability of a compound provided herein for the methods provided herein, including high-performance liquid chromatography/mass spectrometry (HPLC/MS), gas chromatography/mass spectrometry (GC/MS), liquid chromatography/mass spectrometry (LC/MS/MS), and/or capillary electrophoresis (EC).

### 4.5 Pharmaceutical Compositions

Pharmaceutical compositions may be used in the preparation of individual, single unit dosage forms. Pharmaceutical compositions and dosage forms provided herein comprise a compound as described in Section 4.2, such as a compound having a structure of Formula (I), Formula (II), Formula (III), Formula (IV), Formula (V), or of Formula (VI), or a compound selected from Metabolite 1 or Metabolite 2. In certain embodiments, pharmaceutical compositions and dosage forms provided herein comprise one or more of a compound as described in Section 4.2, such as a compound having a structure of Formula (I), Formula (II), Formula (III), Formula (IV), Formula (V), or of Formula (VI), or a compound selected from Metabolite 1 or Metabolite 2. Pharmaceutical compositions and dosage forms can further comprise one or more excipients. Like the amounts and types of excipients, the amounts and specific types of active ingredients in a dosage form may differ depending on factors including, but not limited to, the route by which it is to be administered to subjects. In particular aspects, compositions (e.g., pharmaceutical compositions) described herein can be for in vitro or in vivo uses. Non-limiting examples of uses include uses to improving quality of life and/or energy levels in a subject, and/or to prevent complications associated with anemia, kidney disease or cancer, such as, for example, chronic kidney disease, cardiovascular disease, dyslipidemia, malnutrition, hyperparathyroidism, osteomalacia, and/or adynamic bone disease. The formulations to be used for in vivo administration can be sterile. This is readily accomplished by filtration through, e.g., sterile filtration membranes.

Therapeutic formulations containing a compound as described in Section 4.2 can be prepared for storage by mixing the compound having the desired degree of purity with optional physiologically acceptable carriers, excipients or stabilizers (Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.), in the form of lyophilized formulations or aqueous solutions. Acceptable carriers, excipients, or stabilizers are nontoxic to recipients at the dosages and concentrations employed, and include buffers such as phosphate, citrate, and other organic acids; and/or non-ionic surfactants such as TWEEN™, PLURONICS™ or polyethylene glycol (PEG).

Compositions provided herein can contain one or more of a compound as described in Section 4.2. In one embodiment, a compound as described in Section 4.2, is formulated into suitable pharmaceutical preparations, such as solutions, suspensions, powders, sustained release formulations or elixirs in sterile solutions or suspensions for parenteral administration, or as transdermal patch preparation and dry powder inhalers.

In one embodiment, compositions provided herein are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

Concentrations of a compound as described in Section 4.2 in a pharmaceutical composition provided herein will depend on, e.g., the physicochemical characteristics of the compound, the dosage schedule, and amount administered as well as other factors known to those of skill in the art.

Pharmaceutical compositions described herein are provided for administration to humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, such as tablets, capsules, pills, powders, granules, and oral or nasal solutions or suspensions, and oil-water emulsions containing suitable quantities of a compound as described in Section 4.2. A compound as described in Section 4.2 is, in one embodiment, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of a compound as described in Section 4.2 sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles. Hence, in specific aspects, multiple dose form is a multiple of unit-doses which are not segregated in packaging.

In certain embodiments, a compound as described in Section 4.2 is in a liquid pharmaceutical formulation. Liquid pharmaceutically administrable compositions can, for example, be prepared by dissolving, dispersing, or otherwise mixing a compound as described in Section 4.2 and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md. Dosage forms or compositions containing a compound as described in Section 4.2 in the range of 0.005% to 100% with the balance made up from non-toxic carrier can be prepared.

Pharmaceutical carriers also include ethyl alcohol, polyethylene glycol and propylene glycol for water miscible vehicles; and sodium hydroxide, hydrochloric acid, citric acid or lactic acid for pH adjustment.

In specific embodiments, a compound as described in Section 4.2 can be suspended in micronized or other suitable form. The form of the resulting mixture depends upon a number of factors, including the intended mode of administration and the solubility of the compound in the selected carrier or vehicle.

In other embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a compound as described in Section 4.2 in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. A suitable solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides an example of a formulation. In one embodiment, the resulting solution will be apportioned into vials for lyophilization. Lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier.

A compound as described in Section 4.2 can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,274,552, 6,271,359, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, antibodies described herein are targeted (or otherwise administered) to the visual organs.

In certain embodiments, administration of a compound as described in Section 4.2 may be by topical, oral or parenteral route. In certain embodiments, a compound as described in Section 4.2 may be administered orally, such as in a tablet or capsule formulation.

### 5 EXAMPLES

Deuterium-enriched analogs of the compounds provided herein may generally be prepared according to known procedures for the synthesis of compounds of Formula (I), Formula (II), Formula (III), Formula (IV), Formula (V), and Formula (VI), or a compound selected from Metabolite 1 or Metabolite 2 wherein one or more of the reagents, starting materials, precursors, or intermediates used is replaced by one or more deuterium-enriched reagents, starting materials, precursors, or intermediates. Deuterium-enriched reagents, starting materials, precursors, or intermediates are commercially available or may be prepared by routine procedures known to one of skill in the art. Schemes for the preparation of exemplary deuterium-enriched compounds are illustrated below.

### 5.1 Example 1

The aromatic portions of the compounds of Formula (I), Formula (II), Formula (III), Formula (IV), Formula (V), and Formula (VI), or a compound selected from Metabolite 1 or Metabolite 2 are deuterated by subjecting the compounds of Formula (I), Formula (II), Formula (III), Formula (IV), Formula (V), and Formula (VI), or a compound selected from Metabolite 1 to Metabolite 2 to conditions suitable for aromatic deuteration, which are known in the art, including for example, those disclosed in the following references, each of which are incorporated herein by reference in their entireties: U.S. Publication No. 2007/0255076; U.S. Pat. No. 8,093,422; March, I. “Advanced Organic Chemistry, Reactions, Mechanisms, and Structure,” Sixth Ed., Wiley, New York, 2007; Larsen et al., J. Org. Chem., 1978, 43 (18), pp 3602-3602; Blake et al., J. Chem. Soc., Chem. Commun., 1975, 930; and references cited therein. For example, the compound of Formula (III) is treated with D₂O over 5% Pt/C under hydrogen gas to provide compound A, as depicted in the following scheme.

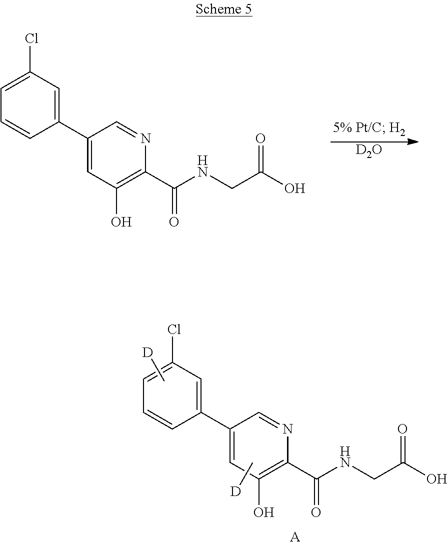

### 5.2 Example 2

A deuterium-enriched glycine portion of the compound of Formula (I) is methylated through the methods of Lanthier et al., as shown in Scheme 6 below.

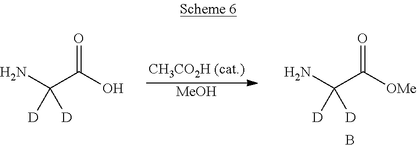

Deuterium-enriched glycine, which is commercially available, is combined with acetic acid and methanol and heated to reflux for 1 hour. The reaction mixture is cooled to room temperature, neutralized with saturated sodium bicarbonate, and the contents are washed with ethyl acetate. The organic phase is isolated and dried over MgSO₄, filtered, and the resulting solvent is concentrated in vacuo to obtain deuterium-enriched compound B.

### 5.3 Example 3

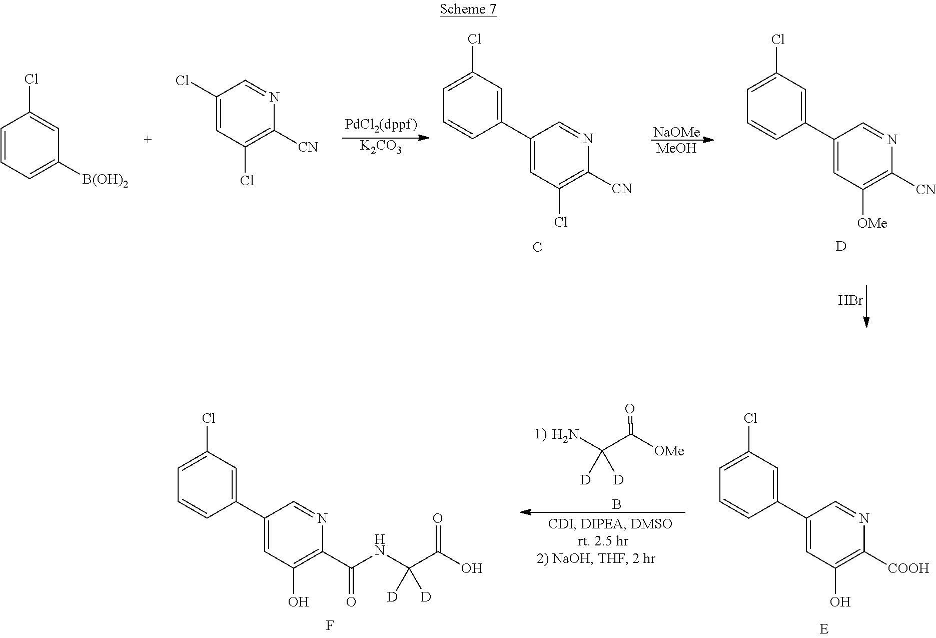

Preparation of Compound C: To a 100-mL round bottom flask adapted for magnetic stirring and equipped with a nitrogen inlet is charged (3-chlorophenyl)boronic acid, 3,5-dichloro-2-cyanopyridine, K₂CO₃, PdCl₂(dppf), dimethylformide, and water. The reaction solution is agitated and heated to 45° C., and held at that temperature for 18 hours after which the reaction is determined to be complete due to the disappearance of 3,5-dichloro-2-cyanopyridine as measured by TLC analysis. The reaction solution is cooled to room temperature and the contents partitioned between ethyl acetate (250 mL) and saturated aqueous NaCl (100 mL). The organic phase is isolated and washed a second time with saturated aqueous NaCl (100 mL). The organic phase is dried over MgSO₄, filtered, and the solvent is concentrated in vacuo. The residue that remained is then slurried in methanol (50 mL) at room temperature for 20 hours. The resulting solid is collected by filtration and washed with cold methanol (50 mL) then hexanes (60 mL) and dried to afford compound C as an admixture containing a 96:4 ratio of the desired regioisomer.

Preparation of Compound D: To a 500 mL round bottom flask adapted for magnetic stirring and fitted with a reflux condenser and nitrogen inlet is charged with compound C, sodium methoxide, and methanol. With stirring, the reaction solution is heated to reflux for 20 hours. The reaction is determined to be complete due to the disappearance of the compound C as measured by TLC analysis. The reaction mixture is cooled to room temperature and combined with water (500 mL), and a solid is formed. The mixture is cooled to 0° C. to 5° C. and stirred for 3 hours. The resulting solid is collected by filtration and washed with water, then hexanes. The resulting cake is dried in vacuo at 40° C. to afford compound D.

Preparation of Compound E: To a 50 mL round bottom flask adapted for magnetic stirring and fitted with a reflux condenser is charged compound D and a 48% aqueous solution of HBr. While being stirred, the reaction solution is heated to reflux for 20 hours. The reaction is determined to be complete due to the disappearance of compound D as determined by TLC analysis. The reaction contents were then cooled to 0° C. to 5° C. with stirring and the pH is adjusted to approximately 2 by the slow addition of 50% aqueous NaOH. Stirring is continued at 0° C. to 5° C. for 3 hours. The resulting solid is collected by filtration and washed with water, then hexanes. The resulting cake is dried in vacuo at 40° C. to afford compound E.

Preparation of Compound F: To a 50 mL round bottom flask adapted for magnetic stirring and equipped with a nitrogen inlet is charged compound E, N,N′-carbonyldiimidazole (CDI), and dimethylsulfoxide. The reaction mixture was stirred at 45° C. for about 1 hour then cooled to room temperature. Compound B is added followed by dropwise addition of diisopropylethylamine. The mixture is then stirred for 2.5 hours at room temperature after which water is added. The contents of the reaction flask is cooled to 0° C. to 5° C. and 1N HCl is added until the solution pH is approximately 2. The solution is extracted with dichloromethane and the organic layer was dried over MgSO₄ for 16 hours. Silica gel is added and the solution slurried for 2 hours after which the solids are removed by filtration. The filtrate is concentrated in vacuo and the resulting residue is slurried in methanol for 2 hours. The resulting solid is collected by filtration, washed with cold methanol, then hexanes. The resulting solid is then combined with tetrahydrofuran and 1M NaOH. The mixture is stirred for 2 hours at room temperature after which it is determined by TLC analysis that the reaction is complete. The reaction solution is adjusted to pH 1 with concentrated HCl, and the solution is heated at 35° C. under vacuum until all the tetrahydrofuran is removed. A slurry forms as the solution is concentrated. With efficient stirring, the pH is adjusted to about 2 with the slow addition of 1M NaOH. The solid which forms is collected by filtration, washed with water, followed by hexanes, then dried under vacuum to afford compound F.

### 6 EQUIVALENTS

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

Other embodiments are within the following claims.

What is claimed is:

1. A deuterium-enriched compound of Formula (II): 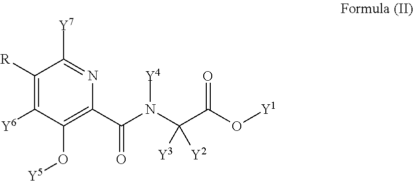
or a pharmaceutically acceptable salt, solvate or hydrate thereof, wherein
R is selected from:
(i) Y⁸; or
(ii) substituted or unsubstituted phenyl;
said substitution selected from:
(i) C(Y⁹⁻¹¹)₃;
(ii) halogen;
(iii) cyano;
(iv) Y¹²;
(v) Y¹³;
(vi) Y¹⁴;
(vii) Y¹⁵; or
(viii) Y¹⁶;
wherein one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and/or Y¹⁶ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and/or Y¹⁶ and/or Y¹⁶ are non- enriched hydrogen atoms.

2. The compound of claim 1, wherein one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and Y¹⁶ is isotopically enriched with deuterium, and the others are non-enriched hydrogens.

3. The compound of claim 1, wherein two of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, and Y¹⁶ are isotopically enriched with deuterium, and the others are non-enriched hydrogens.

4. The compound of claim 1, wherein Y¹, Y⁴, and Y⁵ are hydrogen.

5. A deuterium-enriched compound of Formula (III): 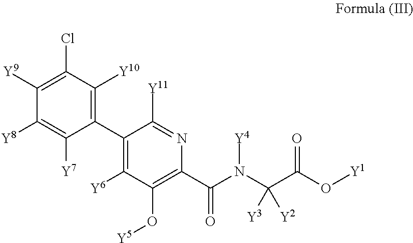
or a pharmaceutically acceptable salt, solvate, hydrate, or stereoisomer thereof, wherein one or more of Y¹, Y², Y³, Y⁴, Y⁵, Y⁶, Y⁷, Y⁸, Y⁹, Y¹⁰, and/or Y¹¹ is a hydrogen that is isotopically enriched with deuterium, and the others of Y¹, Y², Y³, Y⁴, Y⁵, Y⁶, Y⁷, Y⁸, Y⁹, Y¹⁰, and/or Y¹¹ are non-enriched hydrogen atoms.

6. The compound of claim 5, wherein one of Y¹, Y², Y³, Y⁴, Y⁵, Y⁶, Y⁷, Y⁸, Y⁹ Y¹⁰ and Y¹¹ is isotopically enriched with deuterium, and the others are non-enriched hydrogens.

7. The compound of claim 5, wherein two of Y¹, Y², Y³, Y⁴, Y⁵, Y⁶, Y⁷, Y⁸, Y⁹, Y¹⁰, and Y¹¹ are isotopically enriched with deuterium, and the others are non-enriched hydrogens.

8. The compound of claim 5, wherein Y¹, Y⁴, and Y⁵ are hydrogen.

9. A deuterium-enriched compound of Formula (IV): 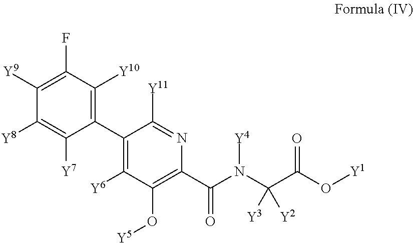
or a pharmaceutically acceptable salt, solvate, hydrate, or stereoisomer thereof,
wherein one or more of Y¹, Y², Y³, Y⁴, Y⁵, Y⁶, Y⁷, Y⁸, Y⁹, Y¹⁰, and/or Y¹¹ is a hydrogen that is isotopically enriched with deuterium, and the others of Y¹, Y², Y³, Y⁴, Y⁵, Y⁶, Y⁷, Y⁸, Y⁹, Y¹⁰, and/or Y¹¹ are non-enriched hydrogen atoms.

10. The compound of claim 9, wherein one of Y¹, Y², Y³, Y⁴, Y⁵, Y⁶, Y⁷, Y⁸, Y⁹, Y¹⁰ and Y¹¹ is isotopically enriched with deuterium, and the others are non-enriched hydrogens.

11. The compound of claim 9, wherein two of Y¹, Y², Y³, Y⁴, Y⁵, Y⁶, Y⁷, Y⁸, Y⁹, Y¹⁰, and Y¹¹ are isotopically enriched with deuterium, and the others are non-enriched hydrogens.

12. The compound of claim 9, wherein Y¹, Y⁴, and Y⁵ are hydrogen.

13. A deuterium-enriched compound of Formula (V): 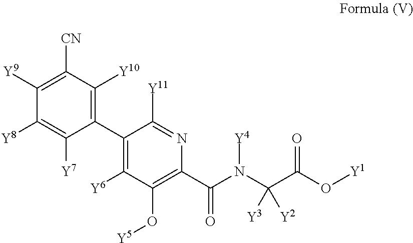
or a pharmaceutically acceptable salt, solvate, hydrate, or stereoisomer thereof,
wherein one or more of Y¹, Y², Y³, Y⁴, Y⁵, Y⁶, Y⁷, Y⁸, Y⁹, Y¹⁰, and/or Y¹¹ is a hydrogen that is isotopically enriched with deuterium, and the others of Y¹, Y², Y³, Y⁴, Y⁵, Y⁶, Y⁷, Y⁸, Y⁹, Y¹⁰, and/or Y¹¹ are non-enriched hydrogen atoms.

14. The compound of claim 13, wherein one of Y¹, Y², Y³, Y⁴, Y⁵, Y⁶, Y⁷, Y⁸, Y⁹, Y¹⁰, and Y¹¹ is isotopically enriched with deuterium, and the others are non-enriched hydrogens.

15. The compound of claim 13, wherein two of Y¹, Y², Y³, Y⁴, Y⁵, Y⁶, Y⁷, Y⁸, Y⁹, Y¹⁰, and Y¹¹ are isotopically enriched with deuterium, and the others are non-enriched hydrogens.

16. The compound of claim 13, wherein Y¹, Y⁴, and Y⁵ are hydrogen.

17. A deuterium-enriched compound of Formula (VI): 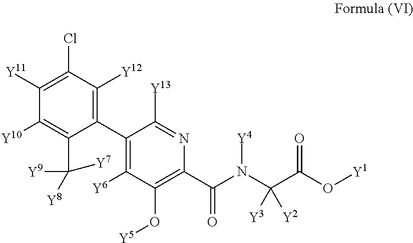
or a pharmaceutically acceptable salt, solvate, hydrate, or stereoisomer thereof,
wherein one or more of Y¹, Y², Y³, Y⁴, Y⁵, Y⁶, Y⁷, Y⁸, Y⁹, Y¹⁰, Y¹¹, Y¹², and/or Y¹³ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², and/or Y¹³ are non-enriched hydrogen atoms.

18. The compound of claim 17, wherein one of Y¹, Y², Y³, Y⁴, Y⁵, Y⁶, Y⁷, Y⁸, Y⁹, Y¹⁰, Y¹¹, Y¹², and Y¹³ is isotopically enriched with deuterium, and the others are non-enriched hydrogens.

19. The compound of claim 17, wherein two of Y¹, Y², Y³, Y⁴, Y⁵, Y⁶, Y⁷, Y⁸, Y⁹, Y¹⁰, Y¹¹, Y¹², and Y¹³ are isotopically enriched with deuterium, and the others are non-enriched hydrogens.

20. The compound of claim 17, wherein Y¹, Y⁴, and Y⁵ are hydrogen.

21. A pharmaceutical composition comprising a compound as in claim 1, or a pharmaceutically acceptable salt, solvate, hydrate, or stereoisomer thereof.

22. The pharmaceutical composition of claim 21, wherein the pharmaceutical composition further comprises a pharmaceutically acceptable carrier.

23. A method for preventing and/or treating kidney disease in a subject, wherein the method comprises administering to the subject a pharmaceutically effective amount of a compound according to claim 1.

24. A method for preventing and/or treating cancer in a subject, wherein the method comprises administering to the subject a pharmaceutically effective amount of a compound according to claim 1.

25. The method of claim 24, wherein the cancer is chosen from Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytoma/Malignant Glioma; Craniopharyngioma; Ependymoblastoma; Ependymoma; Medulloblastoma; Medulloepithelioma; Pineal Parenchymal Tumors of Intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma; Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extra gonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; Intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, Intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; or Wilms Tumor.

26. A method for preventing and/or treating anemia in a subject, wherein the method comprises administering to the subject a pharmaceutically effective amount of a compound according to claim 1.
